SEQUENCE LISTING (52 sequences)

SEQ ID NO: 1 - protein, 178 aa, synthetic construct
  REGION 1..178: note = Human IL-10 Amino Acid Sequence
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - DNA, 1629 bp, synthetic construct
  misc_feature 1..1629: note = Human IL-10 Nucleic Acid Sequence
  source 1..1629: mol_type = other DNA
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 3 - protein, 147 aa, synthetic construct
  REGION 1..147: note = EBV IL-10 Amino Acid Sequence
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 4 - DNA, 632 bp, synthetic construct
  misc_feature 1..632: note = EBV IL-10 Nucleic Acid Sequence
  source 1..632: mol_type = other DNA
tataaatcacttccctatctcaggtaggcctgcacaccttaggtatggagcgaaggttagtggtcactctgcagtgcctggtgctgctttacctggcacctgagtgtggaggtacagaccaatgtgacaattttccccaaatgttgagggacctaagagatgccttcagtcgtgttaaaacctttttccagacaaaggacgaggtagataaccttttgctcaaggagtctctgctagaggactttaagggctaccttggatgccaggccctgtcagaaatgatccaattctacctggaggaagtcatgccacaggctgaaaaccaggaccctgaagccaaagaccatgtcaattctttgggtgaaaatctaaagaccctacggctccgcctgcgcaggtgccacaggttcctgccgtgtgagaacaagagtaaagctgtggaacagataaaaaatgcctttaacaagctgcaggaaaaaggaatttacaaagccatgagtgaatttgacatttttattaactacatagaagcatacatgacaattaaagccaggtgataattccataccctggaagcaggagatgggtgcatttcaccccaaccccccctttcgactgtcatttacaataaa

SEQ ID NO: 5 - protein, 177 aa, synthetic construct
  REGION 1..177: note = CMV IL-10 Amino Acid Sequence
MLSVMVSSSLVLIVFFLGASEEAKPAATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 6 - DNA, 693 bp, synthetic construct
  misc_feature 1..693: note = CMV IL-10 Nucleic Acid Sequence
  source 1..693: mol_type = other DNA
atgctgtcggtgatggtctcttcctctctggtcctgatcgtcttttttctaggcgcttccgaggaggcgaagccggcggcgacgacgacgacgataaagaatacaaagccgcagtgtcgtccggaggattacgcgagcagattgcaagatctccgcgtcacctttcatcgagtaaaacctacgttggtaggtcatgtaggtacggtttattgcgacggtctttcttttccgcgtgtcgggtgacgtagttttcctcttgtagcaacgtgaggacgactactccgtgtggctcgacggtacggtggtcaaaggctgttggggatgcagcgtcatggactggttgttgaggcggtatctggagatcgtgttccccgcaggcgaccacgtctatcctggacttaagacggaattgcatagtatgcgctcgacgctagaatccatctacaaagacatgcggcaatgcgtaagtgtctctgtggcggcgctgtccgcgcagaggtaacaacgtgttcatagcacgctgttttacttttgtcgggctcccagcctctgttaggttgcggagataagtccgtgattagtcggctgtctcaggaggcggaaaggaaatcggataacggcacgcggaaaggtctcagcgagttggacacgttgtttagccgtctcgaagagtatctgcactcgagaaagtag

SEQ ID NO: 7 - protein, 178 aa, synthetic construct
  REGION 1..178: note = Mouse IL-10 Amino Acid Sequence
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 8 - DNA, 1314 bp, synthetic construct
  misc_feature 1..1314: note = Mouse IL-10 Nucleic Acid Sequence
  source 1..1314: mol_type = other DNA
ggggggggggatttagagacttgctcttgcactaccaaagccacaaagcagccttgcagaaaagagagctccatcatgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaaaacacctgcagtgtgtattgagtctgctggactccaggacctagacagagctctctaaatctgatccagggatcttagctaacggaaacaactccttggaaaacctcgtttgtacctctctccgaaatatttattacctctgatacctcagttcccattctatttattcactgagcttctctgtgaactatttagaaagaagcccaatattataattttacagtatttattatttttaacctgtgtttaagctgtttccattggggacactttatagtatttaaagggagattatattatatgatgggaggggttcttccttgggaagcaattgaagcttctattctaaggctggccacacttgagagctgcagggccctttgctatggtgtcctttcaattgctctcatccctgagttcagagctcctaagagagttgtgaagaaactcatgggtcttgggaagagaaaccagggagatcctttgatgatcattcctgcagcagctcagagggttcccctactgtcatcccccagccgcttcatccctgaaaactgtggccagtttgttatttataaccacctaaaattagttctaatagaactcatttttaactagaagtaatgcaattcctctgggaatggtgtattgtttgtctgcctttgtagcagcatctaattttgaataaatggatcttattcg

SEQ ID NO: 9 - protein, 133 aa, synthetic construct [sequence omitted]

SEQ ID NO: 10 - protein, 147 aa, synthetic construct
  REGION 1..147: note = DV07 Amino Acid Sequence
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 11 - DNA, 441 bp, synthetic construct
  misc_feature 1..441: note = DV07 Nucleic Acid Sequence
  source 1..441: mol_type = other DNA
accgaccagtgcgacaacttccctcagatgctgcgggacctgagagatgccttctccagagtgaaaacattcttccagaccaaggacgagctggacaacctgctgctgaaagagtccctgctggaagatttcaagggctacctgggctgtcaggccctgtccgagatgatccagttctacctggaagaagtgatgccccaggccgagaatcaggaccccgagatcaaggaccacgtgaactccctgggcgagaacctgaaaaccctgcggctgagactgcggcggtgccacagatttctgccctgcgagaacaagtccaaggccgtggaacagatcaagaacgccttcaacaagctgcaagagaagggcatctacaaggccatgagcgagttcgacatcttcatcaactacatcgaggcctacatgaccatcaaggccaga

SEQ ID NO: 12 - protein, 567 aa, synthetic construct
  REGION 1..567: note = DV07 Ebo
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGGSFTTTYWNWIRQPPGKGLEWIGEVNYSGNANYNPSLKGRVAISVDTSKNQFSLRLNSVTAADTAIYYCTSRIRSHIAYSWKGDVWGKGTTVTVGGGGSGGGGSGGGGSEIVMTQSPGTLSLSPGERATLSCRASQSVPRNYIGWFQQKPGQAPRLLIYGASSRAAGFPDRFSGSGSGTDFTLTITRLEPEDFAMYYCHQYDRLPYTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 13 - protein, 561 aa, synthetic construct
  REGION 1..561: note = DV07 Ebo EGF
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLRLTCAVYGGSFTNYGVHWIRQPPGKGLEWIGVIWSGGNTDYNTPFTSRVTISVDTSKNQFSLQLNSVTAADTAIYYCTSLTYYDYEFAWGKGTTVTVGGGGSGGGGSGGGGSEIVMTQSPGTLSLSPGERATLSCRASQSIGTNIHWFQQKPGQAPRLLIYYASESISGFPDRFSGSGSGTDFTLTITRLEPEDFAMYYCQQNNNWPTTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 14 - protein, 573 aa, synthetic construct
  REGION 1..573: note = DV07 EboX
  REGION 1..573: note = MISC_FEATURE - DV07 EboX
  REGION 1..573: note = MISC_FEATURE - See specification as filed for detailed description of substitutions and preferred embodiments
  MOD_RES 193..199: note = Any amino acid
  REGION 193..199: note = MISC_FEATURE - This region may encompass 3-7 residues
  MOD_RES 213..230: note = Any amino acid
  REGION 213..230: note = MISC_FEATURE - This region may encompass 14-18 residues
  MOD_RES 263..273: note = Any amino acid
  REGION 263..273: note = MISC_FEATURE - This region may encompass 7-11 residues
  MOD_RES 321..334: note = Any amino acid
  REGION 321..334: note = MISC_FEATURE - This region may encompass 9-14 residues
  MOD_RES 350..358: note = Any amino acid
  REGION 350..358: note = MISC_FEATURE - This region may encompass 5-9 residues
  MOD_RES 391..401: note = Any amino acid
  REGION 391..401: note = MISC_FEATURE - This region may encompass 7-11 residues
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLRLTCAVYGGSFTXXXXXXXWIRQPPGKGLEWIXXXXXXXXXXXXXXXXXXRVTISVDTSKNQFSLQLNSVTAADTAIYYCTSXXXXXXXXXXXWGKGTTVTVGGGGSGGGGSGGGGSEIVMTQSPGTLSLSPGERATLSCXXXXXXXXXXXXXXWFQQKPGQAPRLLIYXXXXXXXXXGFPDRFSGSGSGTDFTLTITRLEPEDFAMYYCXXXXXXXXXXXFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 15 - protein, 569 aa, synthetic construct
  REGION 1..569: note = DV07 EboL3
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGGSFTTTYWNWIRQPPGKGLEWIGEVNYSGNANYNPSLKGRVAISVDTSKNQFSLRLNSVTAADTAIYYCTSRIRSHIAYSWKGDVWGKGTTVTVSSGGGGSGGGGSGGGGSEIVMTQSPGTLSLSPGERATLSCRASQSVPRNYIGWFQQKPGQAPRLLIYGASSRAAGFPDRFSGSGSGTDFTLTITRLEPEDFAMYYCHQYDRLPYTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 16 - protein, 563 aa, synthetic construct
  REGION 1..563: note = DV07 EboEGFL3
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLRLTCAVYGGSFTNYGVHWIRQPPGKGLEWIGVIWSGGNTDYNTPFTSRVTISVDTSKNQFSLQLNSVTAADTAIYYCTSLTYYDYEFAWGKGTTVTVSSGGGGSGGGGSGGGGSEIVMTQSPGTLSLSPGERATLSCRASQSIGTNIHWFQQKPGQAPRLLIYYASESISGFPDRFSGSGSGTDFTLTITRLEPEDFAMYYCQQNNNWPTTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 17 - protein, 565 aa, synthetic construct
  REGION 1..565: note = DEboegfrDV07 Variant 3 (SLP) Amino Acid Sequence
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 18 - DNA, 1698 bp, synthetic construct
  misc_feature 1..1698: note = DEboegfrDV07 Variant 3 (SLP) Nucleic Acid Sequence
  source 1..1698: mol_type = other DNA
accgaccagtgcgacaacttccctcagatgctgcgggacctgagagatgccttctccagagtgaaaacattcttccagaccaaggacgagctggacaacctgctgctgaaagagtccctgctggaagatttcaagggctacctgggctgtcaggccctgtccgagatgatccagttctacctggaagaagtgatgccccaggccgagaatcaggaccccgagatcaaggaccacgtgaactccctgggcgagaacctgaaaaccctgcggctgagactgcggcggtgccacagatttctgccctgcgagaacaagtccaaggccgtggaacagatcaagaacgccttcaacaagctgcaagagaagggcatctacaaggccatgagcgagttcgacatcttcatcaactacatcgaggcctacatgaccatcaaggccagaggcggcggaggatctggcggaggtggaagcggaggcggtggatctcaggttcagttgcagcaatggggcgctggcctgctgaagccttctgagacactgtctctgacctgcgccgtgtacggcttctccctgaccaattatggcgtgcactggatcagacagcctccaggcaaaggcctggaatggctgggagtgatttggagcggcggcaacaccgactacaacacccctttcacctctagagtggccatctccaaggacaactccaagaaccaggtgtccctgagactgaactccgtgaccgctgccgataccgccatctactactgtgctagagccctgacctactacgactacgagttcgcctattggggcaagggcaccaccgtgactgttagtagtggtggtggcggtagtggcggaggcggctcaggcggtggtggatctgaaattgtgctgacccagtctcctggcactctgtctttgagccctggcgagagagctaccctgtcctgtagagcctctcagtccatcggcaccaacatccactggtatcagcagaagcctggacaggcccctcggctgctgattaagtacgcctccgagtccatcagcggcttccctgacagattctccggctctggctctggcaccgacttcaccctgacaatcacccggctggaacctgaggacttcgctatgtactactgccagcagaacaacaactggcccaccacctttggccagggcaccaagctggaaatcaaaggtggcggtggttcaggtggcggaggaagcggcggaggcggatctacagatcagtgtgacaattttccccaaatgctgagggatctgcgggacgccttcagccgggtcaagacattttttcagacaaaggatgaactcgataacctcttgctcaaagagagcctgctcgaggacttcaaaggatatctgggatgccaggctctgagcgaaatgattcagttttatctcgaggaagtcatgcctcaagcagagaaccaggatccagagattaaggatcatgtgaatagcctcggggagaacctcaagacactgagactccggctgagaagatgccaccggtttctgccttgtgaaaacaaaagcaaggctgtcgagcagattaagaatgcttttaacaaattgcaagaaaaagggatctataaggctatgtctgagtttgatatctttatcaattatatcgaagcttatatgactattaaggcccggtag

SEQ ID NO: 19 - protein, 713 aa, synthetic construct
  REGION 1..713: note = DK210egfr Amino Acid Sequence
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 20 - DNA, 2208 bp, synthetic construct
  misc_feature 1..2208: note = DK210egfr Nucleic Acid Sequence
  source 1..2208: mol_type = other DNA
gccgccaccatgggatggtctttgatcctgctgttcctggtggccgtggctaccagagtgcattctaccgaccagtgcgacaacttccctcagatgctgcgggacctgagggacgccttctccagagtgaaaacattcttccagaccaaggacgagctggacaacctgctgctgaaagagtccctgctggaagatttcaagggctacctgggctgtcaggccctgtccgagatgatccagttctacctggaagaagtgatgccccaggccgagaatcaggaccctgagatcaaggaccatgtgaactccctgggcgagaacctgaaaaccctgcggctgagactgcggcggtgtcacagatttctgccctgcgagaacaagtccaaggccgtggaacagatcaagaacgccttcaacaagctgcaagagaagggcatctacaaggccatgagcgagttcgacatcttcatcaactacatcgaggcctacatgaccatcaaggctagaggtggcggaggatctggcggtggtggttctggcggaggcggatctcaggttcagttgcaacaatggggcgctggcctgctgaagccttctgagacactgtctctgacctgcgccgtgtacggcttctctctgaccaattacggcgtgcactggatccggcagccacctggaaaaggactggaatggctgggagtgatttggagcggcggcaacaccgactacaacaccccttttacctctagagtggccatctccaaggacaactccaagaaccaggtgtccctgagactgaactctgtgaccgccgctgataccgccatctactactgtgctagagccctgacctactacgactacgagttcgcttattggggcaagggcaccaccgtgacagtttcatctggcggcggaggaagcggtggcggcggtagcggaggtggtggatctgctcctacctcctccagcaccaagaaaacccagctgcagttggagcatctgctgctggacctccagatgatcctgaacggcatcaacaactacaagaatcccaagctgacccggatgctgaccttcaagttctacatgcccaagaaggccaccgagctgaaacatctgcagtgcctggaagaggaactgaagcccctcgaggaagtgctgaatctggcccagtccaagaacttccacctgaggcctagggacctgatctccaacatcaacgtgatcgtgctcgagctgaagggctccgagacaacctttatgtgcgagtacgccgacgagacagccaccatcgtggaatttctgaaccggtggatcaccttctgccagtccatcatctctacattgaccggtggtggcggatcaggcggtggcggaagcggaggcggaggttctgaaattgtgctgacccagtctcctggcactctgtctttgagtcctggcgagagagctaccctgtcctgcagagcttctcagtccatcggcaccaacatccactggtatcagcagaagcctggacaggcccctcggctgctgattaagtacgcctctgagtccatcagcggcttccctgacagattctctggctctggatctggcaccgacttcaccctgaccatcaccagactggaacccgaggacttcgctatgtactactgccagcagaacaacaactggcccaccacctttggccagggcaccaagttggaaatcaaaggtggcggtggaagtggcggcggtggctcaggcggcggtggatctacagaccagtgtgataattttccacagatgctcagggatctccgcgacgcctttagccgggtcaagacattttttcagacaaaggatgaactcgataatctcctgctcaaagagagcctgctcgaggactttaaaggatacctgggatgccaggctctcagcgaaatgattcagttttatttggaggaagtcatgcctcaagctgaaaaccaggatccagagattaaggatcacgtcaacagcctcggcgagaatctcaagacactgcgcctgaggctgagaagatgccaccggtttctgccttgtgaaaacaagagcaaggctgtcgagcagattaagaatgcttttaacaaattgcaagaaaaagggatctataaggctatgtctgagtttgatatctttatcaattatatcgaagcttatatgactattaaggcccggtga

SEQ ID NO: 21 - protein, 733 aa, synthetic construct
  REGION 1..733: note = DK210her2 (Variant 4)
MGWSLILLFLVAVATRVHSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQEWGAGLLKPSETLSLTCAASGFNIKDTYIHWVRQPPGKGLEWVARIYPTNGYTRYADSVKGRFAISADTSKNQASLRLNSVTAADTAVYYCSRWGGDGFYAMDYWGKGTTVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEIVMTQSPGTLSLSPGERATLSCRASQDVNTAVAWYQQKPGQAPRLLIYSASFLYSGVPDRFSGSRSGTDFTLTITRLEPEDFATYYCQQHYTTPPTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 22 - DNA, 2303 bp, synthetic construct
  misc_feature 1..2303: note = DK210her2 Variant 4 Nucleic Acid Sequence
  source 1..2303: mol_type = other DNA
atcgaaattaatacgactcactatagggagacccaagctggctagcgccgccaccatgggatggtctttgatcctgctgttcctggtggccgtggctaccagagtgcattctaccgaccagtgcgacaacttccctcagatgctgcgggacctgagagatgccttctccagagtgaaaacattcttccagaccaaggacgagctggacaacctgctgctgaaagagtccctgctggaagatttcaagggctacctgggctgtcaggccctgtccgagatgatccagttctacctggaagaagtgatgccccaggccgagaatcaggaccccgagatcaaggaccacgtgaactccctgggcgagaacctgaaaaccctgcggctgagactgcggcggtgccacagatttctgccctgcgagaacaagtccaaggccgtggaacagatcaagaacgccttcaacaagctgcaagagaagggcatctacaaggccatgagcgagttcgacatcttcatcaactacatcgaggcctacatgaccatcaaggccagaggcggcggaggatctggcggaggtggaagcggaggcggtggatctcaggtgcagttgcaagaatggggcgctggcctgctgaagccttccgaaacactgtctctgacctgcgccgccagcggcttcaacatcaaggacacctacatccactgggtccgacagcctccaggcaaaggactggaatgggtcgccagaatctaccccaccaacggctacaccagatacgccgactctgtgaagggcagattcgccatctctgccgacacctccaagaaccaggccagcctgagactgaactctgtgaccgctgctgacaccgccgtgtactactgctctagatggggcggagatggcttctacgccatggactattggggcaagggcaccaccgtgacagttagtagtggtggtggcggtagtggcggaggcggctcaggcggtggtggatctgctcctacatcctccagcaccaagaaaacccagctgcagttggagcatctgctgctggacctccagatgatcctgaacggcatcaacaactacaagaaccccaagctgacccggatgctgaccttcaagttctacatgcccaagaaggccaccgagctgaaacatctgcagtgcctggaagaggaactgaagcccctcgaggaagtgctgaatctggcccagtccaagaacttccacctgaggcctagggacctgatctccaacatcaacgtgatcgtgctcgagctgaagggctccgagacaaccttcatgtgcgagtacgccgacgagacagctaccatcgtggaatttctgaaccggtggatcaccttctgccagtccatcatctctaccctgactggtggcggaggaagcggcggaggcggatctggcggcggaggctctgaaattgtgatgacccagtctcctggcactctgtctctgtctcccggcgagagagctaccctgtcttgtagagccagccaggacgtgaacaccgctgtggcttggtatcagcagaagcctggacaggcccctcggctgctgatctactctgcctcctttctgtactccggcgtgcccgacagattctccggctctagatctggcaccgacttcaccctgaccatcaccagactggaacccgaggacttcgccacctactactgccagcagcactacaccacaccacctacctttggccagggcaccaagctggaaatcaaaggtggtggcggatcaggcggtggcggtagcggtggcggaggttctacagaccagtgtgataattttccccaaatgctgagggatctgcgggacgccttctctagggtcaagacattttttcagacaaaggatgaactcgataacctcttgctcaaagagagcctgctcgaggactttaagggatatctgggatgccaggctctgagcgaaatgattcagttttatttggaggaagtcatgcctcaagcagaaaaccaggatccagagattaaggatcatgtcaacagcctcggcgagaatctcaagacactgcgcctgaggctgagaagatgccaccggtttctgccttgtgaaaacaaaagcaaggctgtcgagcagattaagaatgcttttaacaaactccaagaaaaagggatctataaggctatgtctgagtttgatatctttatcaattatatcgaagcttatatgactattaaggctcgctaggggcccgtttaaacccgctgatcagcctcgactgtgccttctagtt

SEQ ID NO: 23 - protein, 733 aa, synthetic construct
  REGION 1..733: note = DK210her2 (Variant 5)
MGWSLILLFLVAVATRVHSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQEWGAGLLKPSETLSLTCAASGFNIKDTYIHWVRQPPGKGLEWVARIYPTNGYTRYADSVKGRFAISADTSKNQASLQMNSLRAEDTAVYYCSRWGGDGFYAMDYWGKGTTVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEIQMTQSPSSLSLSPGERATLSCRASQDVNTAVAWYQQKPGQAPRLLIYSASFLYSGVPDRFSGSRSGTDFTLTISSLQPEDFATYYCQQHYTTPPTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 24 - DNA, 2303 bp, synthetic construct
  misc_feature 1..2303: note = DK210her2 Variant 5 Nucleic Acid Sequence
  source 1..2303: mol_type = other DNA
atcgaaattaatacgactcactatagggagacccaagctggctagcgccgccaccatgggatggtctttgatcctgctgttcctggtggccgtggctaccagagtgcattctaccgaccagtgcgacaacttccctcagatgctgcgggacctgagagatgccttctccagagtgaaaacattcttccagaccaaggacgagctggacaacctgctgctgaaagagtccctgctggaagatttcaagggctacctgggctgtcaggccctgtccgagatgatccagttctacctggaagaagtgatgccccaggccgagaatcaggaccccgagatcaaggaccacgtgaactccctgggcgagaacctgaaaaccctgcggctgagactgcggcggtgccacagatttctgccctgcgagaacaagtccaaggccgtggaacagatcaagaacgccttcaacaagctgcaagagaagggcatctacaaggccatgagcgagttcgacatcttcatcaactacatcgaggcctacatgaccatcaaggccagaggcggcggaggatctggcggaggtggaagcggaggcggtggatctcaggtgcagttgcaagaatggggcgctggcctgctgaagccttccgaaacactgtctctgacctgcgccgccagcggcttcaacatcaaggacacctacatccactgggtccgacagcctccaggcaaaggactggaatgggtcgccagaatctaccccaccaacggctacaccagatacgccgactctgtgaagggcagattcgccatctctgccgacacctccaagaaccaggccagcctgcagatgaacagcctgagagctgaggacaccgccgtgtactactgctctagatggggcggagatggcttctacgccatggactattggggcaagggcaccaccgtgacagttagtagtggtggtggcggtagtggcggaggcggctcaggcggtggtggatctgctcctacatcctccagcaccaagaaaacccagctgcagttggagcatctgctgctggacctccagatgatcctgaacggcatcaacaactacaagaaccccaagctgacccggatgctgaccttcaagttctacatgcccaagaaggccaccgagctgaaacatctgcagtgcctggaagaggaactgaagcccctcgaggaagtgctgaatctggcccagtccaagaacttccacctgaggcctagggacctgatctccaacatcaacgtgatcgtgctcgagctgaagggctccgagacaaccttcatgtgcgagtacgccgacgagacagctaccatcgtggaatttctgaaccggtggatcaccttctgccagtccatcatctctaccctgactggtggcggaggaagcggcggaggcggatctggcggcggaggctctgaaattcagatgacccagtctccttccagcctgtctctgtcccctggcgagagagctaccctgtcttgtagagccagccaggacgtgaacaccgctgtggcttggtatcagcagaagcctggacaggcccctcggctgctgatctactctgcctcctttctgtactccggcgtgcccgacagattctccggctctagatctggcaccgactttaccctgacaatcagctccctgcagcctgaggacttcgccacctactactgccagcagcactacaccacaccacctacctttggccagggcaccaagctggaaatcaaaggtggtggcggatcaggcggtggcggtagcggtggcggaggttctacagaccagtgtgataattttccccaaatgctgagggatctgcgggacgccttctctagggtcaagacattttttcagacaaaggatgaactcgataacctcttgctcaaagagagcctgctcgaggacttcaaaggatatctgggatgccaggctctgagcgaaatgattcagttttatttggaggaagtcatgcctcaagcagaaaaccaggatccagagattaaggatcatgtcaacagcctcggcgagaatctcaagacactgagactgaggctgcggagatgtcaccggtttctgccttgtgaaaacaagagcaaggctgtcgagcagattaagaatgcttttaacaaactccaagaaaaagggatctataaggctatgtctgagtttgatatctttatcaattatatcgaagcttatatgactattaaggctcgctaggggcccgtttaaacccgctgatcagcctcgactgtgccttctagtt

SEQ ID NO: 25 - protein, 733 aa, synthetic construct
  REGION 1..733: note = DK12DV07 EGFR DEK005
MGWSLILLFLVAVATRVHSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGFNIKDTYIHWIRQPPGKGLEWIGRIYPTNGYTRYADSVKGRVAISVDTSKNQFSLRLNSVTAADTAIYYCTSWGGDGFYAMDYWGKGTTVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEIVMTQSPGTLSLSPGERATLSCRASQDVNTAVAWFQQKPGQAPRLLIYSASFLYSGFPDRFSGSGSGTDFTLTITRLEPEDFAMYYCQQHYTTPPTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 26 - protein, 1090 aa, synthetic construct
  REGION 1..1090: note = DEK0005: 22AASKEC_3253190_DK1210(EGFR)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 27 - protein, 1090 aa, synthetic construct
  REGION 1..1090: note = DKd12DV07 EGFR DEK006
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 28 - protein, 941 aa, synthetic construct
  REGION 1..941: note = DKm12DV07 hEGFR DEK0014
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSDCDIEGKDGKQYESVLMVSIDQLLDSMKEIGSNCLNNEFNFFKRHICDANKEGMFLFRAARKLRQFLKMQSTGDFDLHLLKVSEGTTILLQCTGQVKGRKPAALGEAQPTKSLEEQKSLKEQKKLNDLCFLKRLLQEIKTCWNKILMGTKEHGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 29 - protein, 1110 aa, synthetic construct
  REGION 1..1110: note = DKa12DV07 EGFR L512L310 DEK0039
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 30 - protein, 1118 aa, synthetic construct
  REGION 1..1118: note = DKa12DV07 EGFR L512 DEK0031
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 31 - protein, 1130 aa, synthetic construct
  REGION 1..1130: note = DKa12DV07 EGFR L51210 DEK0040
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 32 - protein, 1100 aa, synthetic construct
  REGION 1..1100: note = DKa12DV07 EGFR L412 DEK0071
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 33 - protein, 1091 aa, synthetic construct
  REGION 1..1091: note = DK12DV07 Her2 DEK0017
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSQVQLQQWGAGLLKPSETLSLTCAVYGFNIKDTYIHWIRQPPGKGLEWLGRIYPTNGYTRYADSVKGRVAISKDNSKNQVSLRLNSVTAADTAIYYCARWGGDGFYAMDYWGKGTTVTVSSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSEIVLTQSPGTLSLSPGERATLSCRASQDVNTAVAWYQQKPGQAPRLLIKSASFLYSGFPDRFSGSGSGTDFTLTITRLEPEDFAMYYCQQHYTTPPTFGQGTKLEIKGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 34 - protein, 1112 aa, synthetic construct
  REGION 1..1112: note = DKa12DV07 CD20 DEK0041
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSGGGGSGGGGSQAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKGGGGSGGGGSGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 35 - protein, 1132 aa, synthetic construct
  REGION 1..1132: note = DKa12DV07 CD20 L51210 DEK0078
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKQKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKQASISVRAQDRYYSSSWSEWASVPCSGGGGSGGGGSGGGGSGGGGSGGGGSQAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSSGGGGSGGGGSGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSGGGGSGGGGSQIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKGGGGSGGGGSGGGGSGGGGSGGGGSRNLPVATPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 36 - protein, 732 aa, synthetic construct
  REGION 1..732: note = DK7DV07 EGFR
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGFSLTNYGVHWIRQPPGKGLEWLGVIWSGGNTDYNTPFTSRVAISKDNSKNQVSLRLNSVTAADTAIYYCARALTYYDYEFAYWGKGTTVTVSSGGGGSGGGGSGGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGGSGGGGSGGGGSEIVLTQSPGTLSLSPGERATLSCRASQSIGTNIHWYQQKPGQAPRLLIKYASESISGFPDRFSGSGSGTDFTLTITRLEPEDFAMYYCQQNNNWPTTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 37 - protein, 721 aa, synthetic construct
  REGION 1..721: note = DK7DV07 HER2
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGSGGGSQVQLQQWGAGLLKPSETLSLTCAVYGFNIKDTYIHWIRQPPGKGLEWLGRIYPTNGYTRYADSVKGRVAISKDNSKNQVSLRLNSVTAADTAIYYCARWGGDGFYAMDYWGKGTTVTVSSGGGSGGGSGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGSGGGSGGGSEIVLTQSPGTLSLSPGERATLSCRASQDVNTAVAWYQQKPGQAPRLLIKSASFLYSGFPDRFSGSGSGTDFTLTITRLEPEDFAMYYCQQHYTTPPTFGQGTKLEIKGGGSGGGSGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 38 - protein, 722 aa, synthetic construct
  REGION 1..722: note = DK7DV07 CD20
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGSGGGSGGGSQAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSSGGGSGGGSGGGSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGGGSGGGSGGGSQIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKGGGSGGGSGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 39 - protein, 7 aa, synthetic construct
  REGION 1..7: note = Linker 1
SSGGGGS

SEQ ID NO: 40 - protein, 17 aa, synthetic construct
  REGION 1..17: note = Linker
SSGGGGSGGGGSGGGGS

SEQ ID NO: 41 - protein, 10 aa, synthetic construct
  REGION 1..10: note = Linker Lx2
GGGGSGGGGS

SEQ ID NO: 42 - protein, 15 aa, synthetic construct
  REGION 1..15: note = Linker x3
GGGGSGGGGSGGGGS

SEQ ID NO: 43 - protein, 20 aa, synthetic construct
  REGION 1..20: note = Linker x4
GGGGSGGGGSGGGGSGGGGS

SEQ ID NO: 44 - protein, 25 aa, synthetic construct
  REGION 1..25: note = Linker Lx5
GGGGSGGGGSGGGGSGGGGSGGGGS

SEQ ID NO: 45 - protein, 219 aa, Homo sapiens
  REGION 1..219: note = MISC_FEATURE - IL-12 p35
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 46 - protein, 328 aa, Homo sapiens
  REGION 1..328: note = MISC_FEATURE - IL-12 p40
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 47 - protein, 243 aa, Homo sapiens
  REGION 1..243: note = MISC_FEATURE - IL-27 p28
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 48 - DNA, 1161 bp, Homo sapiens
  misc_feature 1..1161: note = IL-27 EBI3
  source 1..1161: mol_type = other DNA
gaattccgcagccatgaccccgcagcttctcctggcccttgtcctctgggccagctgcccgccctgcagtggaaggaaagggcccccagcagctctgacactgccccgggtgcaatgccgagcctctcggtacccgatcgccgtggattgctcctggaccctgccgcctgctccaaactccaccagccccgtgtccttcattgccacgtacaggctcggcatggctgcccggggccacagctggccctgcctgcagcagacgccaacgtccaccagctgcaccatcacggatgtccagctgttctccatggctccctacgtgctcaatgtcaccgccgtccacccctggggctccagcagcagcttcgtgcctttcataacagagcacatcatcaagcccgaccctccagaaggcgtgcgcctaagccccctcgctgagcgccacgtacaggtgcagtgggagcctcccgggtcctggcccttcccagagatcttctcactgaagtactggatccgttacaagcgtcagggagctgcgcgcttccaccgggtggggcccattgaagccacgtccttcatcctcagggctgtgcggccccgagccaggtactacgtccaagtggcggctcaggacctcacagactacggggaactgagtgactggagtctccccgccactgccacaatgagcctgggcaagtagcaagggcttcccgctgcctccagacagcacctgggtcctcgccaccctaagccccgggacacctgttggagggcggatgggatctgcctagcctgggctggagtccttgctttgctgctgctgagctgccgggcaacctcagatgaccgacttttccctttgagcctcagtttctctagctgagaaatggagatgtactactctctcctttacctttacctttaccacagtgcagggctgactgaactgtcactgtgagatattttttattgtttaattagaaaagaattgttgttgggctgggcgcagtggatcgcacctgtaatcccagtcactgggaagccgacgtgggtgggtagcttgaggccaggagctcgaaaccagtccgggccacacagcaagaccccatctctaaaaaattaatataaatataaaataaaaaaaaaaaaaaggaattc

SEQ ID NO: 49 - protein, 188 aa, Homo sapiens [sequence omitted]

SEQ ID NO: 50 - protein, 177 aa, Homo sapiens
  REGION 1..177: note = MISC_FEATURE - IL-7
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 51 - protein, 162 aa, Homo sapiens
  REGION 1..162: note = MISC_FEATURE - IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 52 - protein, 162 aa, Homo sapiens
  REGION 1..162: note = MISC_FEATURE - IL-21
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

(19) United States
(12) Patent Application Publication
(10) Pub. No.: US 2023/0210953 A1
(43) Pub. Date: Jul. 6, 2023

(54) USE OF DUAL CYTOKINE FUSION PROTEINS COMPRISING IL-10 AND ADOPTIVE CELL THERAPIES OR BISPECIFIC T-CELL ENGAGERS TO TREAT CANCER

(71) Applicant: Deka Biosciences, Inc., Germantown, MD (US)

(72) Inventor: John MUMM, Germantown, MD (US)

(21) Appl. No.: 18/067,535

(22) Filed: Dec. 16, 2022

Related U.S. Application Data
(60) Provisional application No. 63/265,521, filed on Dec. 16, 2021.

(51) Int. Cl. A61K 38/20; A61K 38/17; A61K 35/17; A61K 39/395; A61P 35/00

(52) U.S. Cl. CPC A61K 38/2066; A61K 38/1774; A61K 35/17; A61K 38/2013; A61K 39/39558; A61P 35/00

(57) ABSTRACT

The application relates to a method of treating cancer or a tumor comprising administering a dual cytokine fusion protein composition, pharmaceutical composition, and/or formulation thereof comprising IL-10 or IL-10 variant molecules fused to a single chain variable fragment scaffolding system and a second cytokine, where the second cytokine is linked in the hinge region of the scFv, in combination with an adaptive cell therapies or bispecific T cell engagers.

### CROSS-REFERENCE TO RELATED APPLICATION

The present application claims priority to U.S. Provisional Patent Application Ser. No. 63/265,521 filed on Dec. 16, 2021, the content of which is incorporated herein by reference in its entirety.

### REFERENCE TO AN ELECTRONIC SEQUENCE LISTING

The contents of the electronic sequence listing (039451-00084-Sequence-Listing.xml; Size: 107,499 bytes; and Date of Creation: Dec. 16, 2022) are herein incorporated by reference in its entirety.

### FIELD OF INVENTION

The present disclosure relates to the field of biotechnology, and more specifically, to the use of a novel dual cytokine fusion protein, called Diakines, comprising Interleukin-10 (“IL-10”) and Interleukin-2 (“IL-2”) in combination with conventional Adoptive Cell Therapies (“ACTs”) or Bispecific T-cell Engagers (“BiTE”) therapies to treat cancer.

### INTRODUCTION

IL-10, originally named cytokine synthesis inhibitory factor (Malefyt, Interleukin 10 inhbits cytokine synthesis by human monocytes: An autoreglatory role of IL-10 produced by monocytes, 1991), is a pleiotropic cytokine known to both suppress inflammatory response (Fedorak, 2000), and more recently activate CD8⁺ T cells to induce Interferon γ (“IFNγ”) dependent anti-tumor immune responses (Mumm J., 2011). IL-10 is a non-covalent homo-dimeric cytokine with structural similarities to IFNγ. IL-10 binds to the IL-10 receptor, which consists of two subunits of the IL10 receptor 1 (IL10R1) and two subunits of the IL-10 receptor 2 (IL10R2) (Moore, 2001). The IL-10 receptor complex is expressed on the surface of most hematopoietic cells and most highly expressed on macrophages and T-cells. While IL-10 has been reported to be both an immunosuppressive (Schreiber, 2000) and an immunostimulatory cytokine (Mumm, 2011), clinical evaluation of IL-10 treatment of Crohn's patients resulted in an inverse dose response (Fedorak, 2000; Schreiber, 2000), whereas treatment of cancer patients with PEGylated IL-10 resulted in dose titratable potent anti-tumor responses (Naing, 2018). PEGylated IL-10 anti-tumor response requires endogenous CD8+ T cells and IFNγ (Mumm, 2011). Treatment of tumor bearing animals with PEGylated IL-10 results in increased intratumor CD8+ T cells and increased IFNγ on a per cell basis. Most recently, however, cancer patients treated with PEGylated IL-10 lead to evidence of immune stimulation, but no increase in anti-tumor responses (Spigel, 2020).

Interleukin-2 (“IL-2”) is a four-helix bundle pleiotropic cytokine known to induce anti-tumor immune responses (Jiang, 2016), but also exhibiting high toxicity due to uncontrolled activation of and secretion of IFNγ by natural killer (“NK”) cells and CD4⁺ T cells and expansion of T regulatory cells (Chinen, 2016). For this reason, many groups have attempted to mutate IL-2 to reduce its binding to the high affinity receptor, resulting in reduced IL-2 toxicity (Chen, 2018). These muteins have not generated substantial clinical success (Bentebibe, 2019), which suggests other mechanisms need to be employed to reduce the potentially lethal toxicity of IL-2.

IL-10 has been reported to suppress IL-2 driven IFNγ production secreted by both NK and CD4⁺ T cells (Scott, 2006), but it has also been reported to act as a cofactor for IL-2 induced CD8⁺ T cell proliferation (Groux, 1998). It is therefore not known whether IL-2 and IL-10 will co-activate cells of the immune system or cancel each other out.

It was surprisingly discovered that Epstein-Barr virus (“EBV”) IL-10 variants with one or more amino acid substitutions (at amino acid position 31, 75, or at both positions of the mature EBV IL-10 amino acid sequence of SEQ ID No. 3) in key IL-10 receptor binding domain regions, altered the ability of EBV IL-10 to bind to and activate the IL-10 receptor. These modifications included the ability to increase the affinity of EBV IL-10 for the IL-10 receptor. The inventor discovered that EBV IL-10 variant molecules act as IL-10 receptor agonists capable of treating immune diseases, inflammatory diseases or conditions, and in treating cancer. The high affinity EBV IL-10 variant (which comprises two amino acid substitutions at positions 31 and 75 termed “DV07”), when incorporated as monomers into a single chain variable region (scFv) scaffolding system comprising non-immunogenic variable heavy (“VH”) and variable light (“VL”) regions, resulted in a half-life extended molecule that properly folded and remained functionally active. See, U.S. Pat. Nos. 10,858,412; 10,975,133; 10,981,965; 10,975,134; and 10,981,966 each incorporated by reference in its entirety; see, also FIG. 1 as a representative schematic diagram. The EBV IL-10 variants incorporated into the scaffolding system showed enhanced IL-10 function on both inflammatory cells (e.g., monocytes/macrophages/dendritic cells) and immune cells (e.g., CD8⁺ T-cells). Id. In one iteration, the scFv scaffolding system utilized VH and VL regions originally obtained from a human anti-ebola antibody. The single cytokine fusion protein was termed DeboDV07. Id. The inventor also found that IL-10 functionality was not disrupted when the 6 complementarity-determining regions (“CDRs”) from the VH and VL regions of the scFv originally obtained from the human anti-ebola antibody were engrafted (i.e., replacing the 6 CDRs from one antibody with 6 CDRs from a second antibody) with 6 CDRs from antibodies recognizing various tumor associated antigen (“TAA”), such as anti-EGFR, anti-VEGFR1, anti-VEGFR2, and anti-HER2 antibodies. See, U.S. Pat. Nos. 10,858,412; 10,975,133; 10,981,965; 10,975,134; and 10,981,966.

The inventor improved DeboDV07 by incorporating a second cytokine into the single cytokine fusion protein (described above; see, also co-pending U.S. application Ser. No. 17/199,239, filed Mar. 11, 2021, incorporated by reference in its entirety). Specifically, a second cytokine is incorporated in the linker region between the VH and VL region of a scFv. See, e.g., Id. at FIG. 2 (a representative schematic diagram of the dual cytokine fusion protein). The dual cytokine fusion protein is capable of delivering both IL-10 and another cytokine (such as but not limited to IL-2) to specific TAAs when engrafted with CDRs from various TAA targeting monoclonal antibodies. In the present application, the inventor describes a novel use of the dual cytokine fusion protein, which are known as Diakines™ (“DK”), in bolstering other known immunotherapeutic modalities, such as Adoptive Cell Therapies (“ACT”), including but not limited to Chimeric Antigen Receptor T-cell (“CAR-T”), engineered T-Cell Receptor T-cell (“TCR-T”), Natural Killer (“NK”) cells and/or Tumor-Inflitrating Lymphocytes (“TILs”), and Bispecific T-cell Engagers (“BiTE”). As used in the present application the term “diakine” or “diakines” is a generic term that refers to a novel class of dual cytokine fusion proteins linked to together on a targeted and half-life extending scFv.

With respect to CAR-T cell therapies, the therapies have exhibited significant success in hematological malignancies, but there have been limited equivalent examples when using these CAR-T cells in solid tissue tumors (Ma, 2019; Castellarin, 2018; Wagner, 2020).

The challenges with the current approach appear to share similarities with most immune stimulatory therapies. Specifically, the challenges include off target toxicity (Bonifant, 2016; Bianca Santomasso, 2019), CAR-T cell persistence (Jafarzadeh, 2020; Christopher DeRenzo, 2019), and the ability to infiltrate the tumor microenvironment (“TME”) (Rodriguez-Garcia, 2020; Zou, 2019).

Both IL-2 (Groux, 1998; Ross, 2018), and IL-10 (Berman, 1996; Chan, 2015; Naing A., 2018; Naing A., Safety, Antitumor Activity, and Immune Activation of Pegylated Recombinant Human Interleukin-10 (AM0010) in Patients With Advanced Solid Tumors, 2016; Emmerich, 2012; Mumm J., 2011), specifically enhance anti-tumor T cell function. However, while Pegylated IL-10 has been reported to enhance CAR-T function (McCauley, 2018) use of IL-2 in combination with CAR-T cell therapy is fraught with challenges, primarily due to IL-2 related toxicity (Tang, 2018).

Accordingly, the present application overcomes this issue by using, in one aspect, a diakine comprising both (1) IL2 and IL10, termed DK2¹⁰, that controls IL-2 mediated toxicity, (2) IL12 and IL10, (3) IL7 and IL10, or (4) IL15 and IL-10. By enriching the coupled IL-2 and IL10 into the tumor vasculature via targeting to a TAA, such as EGFR2, HER2, or VEGFR2 (Smith, 2010), to name a few, DK2¹⁰ will drive TME activation of CAR-T cells, enabling activation, infiltration and persistence while limiting toxicity both through tumor specific activation of the CAR-T and directly suppressing cytokine release syndrome and IL-2 toxicity by DV07. The same premise is also believed to be promising for ACT therapies and BiTEs.

### SUMMARY OF VARIOUS ASPECTS OF THE INVENTION

The present disclosure generally relates to a method of using a dual cytokine fusion protein, termed a diakine, in combination with conventional immunotherapies.

Thus in a first aspect, the present disclosure relates to a method of using a diakine, comprising IL-10 or various IL-10 variants, a half-life extending targeting domain, and a second cytokine, in combination with conventional immunotherapies, including but not limited to, engineered immune cells (such as CAR-T cells, TCR-T cells, TILs, or NK cells) or BiTEs. In certain embodiments, the method uses a diakine comprising an IL-10, such as but not limited to human, mouse, cytomegalovirus, (“CMV”), or EBV IL-10 forms or IL-10 variant molecules thereof, wherein the IL-10 variant has one or more amino acid substitution(s) that impact the IL-10 receptor binding domains. In certain embodiments, the method uses a diakine comprising an IL-10, IL-12 or IL-27 or variants thereof. Each of the aforementioned diakine varieties—comprising IL-10 IL-12, or IL-27—will also include a second cytokine, which is a cytokine that is different from the first cytokine and works in tandem with the IL-10, IL-12 or IL-27 or variants thereof, such that there is an additive or synergistic effect when the first and second cytokines are coupled and targeted together to a specific antigen by the half-life extending antigen targeting domain of the diakine. These second cytokines include, amongst others, IL-6, IL-4, IL-1, IL-2, IL-3, IL-5, IL-7, IL-8, IL-9, IL-15, IL-21 IL-26, IL-27, IL-28, IL-29, GM-CSF, G-CSF, IFN-α, IFN-β, IFN-γ, TGF-β, or TNF-α, TNF-β, basic FGF, EGF, PDGF, IL-4, IL-11, or IL-13, preferably IL-2. The antigen targeting domain of the diakine comprises a targeting domain selected from an antibody, antibody fragment (e.g., scFv, antigen binding fragment), or antigen binding portion that directs the diakine to a target antigen recognized by the variable heavy (VH) and variable light (VL) chain regions of the antibody, antibody fragment, or antigen binding portion thereof. In certain embodiments, the antigen targeting domain is a scFv. In certain aspects, the scFv has specificity for a tumor associated antigen (TAA), the TAA being selected from a variety of antigenic targets found on the surface of solid or hematological tumors. In one aspect, the antigen targeting domain is a scFv comprising 3 CDRs in the VH and 3 CDRs in the VL region of the scFv. In another aspect, the scFv may be engrafted with 3 CDRs in the VH and 3 CDRs in the VL from another antibody, but retaining the original VH and VL framework regions. In other embodiments, the engineered cell includes a recombinant antigen receptor, such as but not limited to a CAR, a T cell receptor (“TCR”) or a functional non-TCR, preferably a CAR that specifically targets a tumor associated antigen (TAA). In certain embodiments, the engineered cell is a T-cell.

In yet another aspect, the present disclosure relates to a method of using a diakine of formula (I) in combination with a TAA targeting engineered immune cell or a BiTE, wherein formula (I) is:

NH₂-(IL10)-(X¹)—(Zₙ)—(X²)—(IL10)-COOH (Formula I);

wherein
“IL10” is a monomer of IL-10, wherein the IL-10 is human, mouse, CMV, or EBV IL-10, or a variant thereof, more preferably a IL10 is monomer comprising a sequence selected from SEQ ID Nos: 1, 3, 7, 9, or 10,
“X¹” is a VL or VH region obtained from a first monoclonal antibody; “X²” is a VH or VL region obtained from the first monoclonal antibody; wherein when X¹ is a VL, X² is a VH or when X¹ is a VH, X² is a VL, preferably wherein the VH and VL regions are a scFv obtained from a human anti-ebola antibody, and the VH and VL regions are engrafted with 6 CDRs (3 from the VH and 3 from the VL) from a second antibody;
“Z” is any cytokine other than IL-10, preferably IL-6, IL-4, IL-1, IL-2, IL-3, IL-5, IL-7, IL-8, IL-9, IL-15, IL-21 IL-26, IL-27, IL-28, IL-29, GM-CSF, G-CSF, IFN-α, IFN-β, IFN-γ, TGF-β, or TNF-α, TNF-β, basic FGF, EGF, PDGF, IL-4, IL-11, or IL-13; and
“n” is an integer selected from 1-2,
   wherein the second antibody is VEGFR2, CD3, CD4, CD5, CD7, CD19, CD20, CD22, CD25, CD30, CD33, CD34, CD38, CD40, CD44, CD52, CD56, CD70, CD79B, CD117, CD123, CD138, CD147, IL-22R1, B cell maturation antigen (BCMA), C-type lectin-like molecule-1 (CLL01), CD5, CD147, latent membrane protein 1 (LMP-1), signaling lymphocytic activation molecule F7 (SLAMF7), NY-ESO-1, transmembrane activator and CAML interactor (TACI), CS-1, CXCR4, NKG2D, B7-H3, EGFR, PD-1, PDL-1, HER2, HER3, EpCAM, PSCA, MUC1, Lewis-Y, GPC3, AXL, Claudin18.2, GD2, CTLA-4, CEA, PDGFR, mesothelin (MESO), PSCA, PSMA, BCMA, or PSA.

In yet another aspect, the present disclosure relates to a method of using an IL-10 diakine of formula (II) in combination with a TAA targeting immune cell or a BiTE

NH₂-(IL10)-(L)-(X¹)-(L)-(Zₙ)-(L)-(X²)-(L)-(IL10)-COOH (Formula II);
wherein
“IL-10” is a monomer sequence selected from SEQ ID Nos: 1, 3, 7, 9, or 10;
“L” is any linker, more preferably the linker is selected from SEQ ID No: 39, 40, or 41;
“X¹” is a VL or VH region obtained from a first monoclonal antibody; “X²” is a VH or VL region obtained from the first monoclonal antibody; wherein when X¹ is a VL, X² is a VH or when X¹ is a VH, X² is a VL, preferably wherein the VH and VL regions are a scFv obtained from a human anti-ebola antibody, and the VH and VL regions are engrafted with 6 CDRs (3 from the VH and 3 from the VL) from a second antibody, wherein the second antibody is VEGFR2, CD3, CD4, CD5, CD7, CD19, CD20, CD22, CD25, CD30, CD33, CD34, CD38, CD40, CD44, CD52, CD56, CD70, CD79B, CD117, CD123, CD138, CD147, IL-22R1, BCMA, CLL01, CD5, CD147, ILMP-1, SLAMF7, NY-ESO-1, TACI, CS-1, CXCR4, NKG2D, B7-H3, EGFR, PD-1, PDL-1, HER2, HER3, EpCAM, PSCA, MUC1, Lewis-Y, GPC3, AXL, Claudin18.2, GD2, CTLA-4, CEA, PDGFR, MESO, PSCA, PSMA, BCMA, or PSA;
“Z” is a cytokine selected from IL-6, IL-4, IL-1, IL-2, IL-3, IL-5, IL-7, IL-8, IL-9, IL-15, IL-21 IL-26, IL-27, IL-28, IL-29, GM-CSF, G-CSF, IFN-α, IFN-β, IFN-γ, TGF-β, or TNF-α, TNF-β, basic FGF, EGF, PDGF, IL-4, IL-11, or IL-13, preferably IL-2; and
“n” is an integer selected from 1-2.

In other aspects, the present disclosure relates to a method of treating cancer comprising administering to a subject in need thereof, an effective amount of the diakine, preferably a DK2¹⁰, DK7¹⁰, DK12¹⁰, DK15¹⁰ DK21¹⁰, DK27¹⁰, DKIFNa¹⁰ in combination with an engineered immune cells, preferably CAR-T therapy, or a BiTE, wherein the DK2¹⁰, DK7¹⁰, DK12¹⁰, DK15¹⁰ DK21¹⁰, DK27¹⁰, or DKIFNa¹⁰ includes and anti-ebola scaffolding system (which comprises a VH/VL pair or scFv) engrafted with CDRs from an antibody with specificity for VEGFR2, CD3, CD4, CD5, CD7, CD19, CD20, CD22, CD25, CD30, CD33, CD34, CD38, CD40, CD44, CD52, CD56, CD70, CD79B, CD117, CD123, CD138, CD147, IL-22R1, BCMA, CLL01, CD5, CD147, ILMP-1, SLAMF7, NY-ESO-1, TACI, CS-1, CXCR4, NKG2D, B7-H3, EGFR, PD-1, PDL-1, HER2, HER3, EpCAM, PSCA, MUC1, Lewis-Y, GPC3, AXL, Claudin18.2, GD2, CTLA-4, CEA, PDGFR, MESO, PSCA, PSMA, BCMA, or PSA.

In another aspect, the present disclosure relates to a method of using an IL-12 diakine of formula (III) in combination with a TAA targeting engineered immune cell or a BiTE

NH₂—(R¹)—(X¹)—(Zₙ)—(X²)—(R²)—COOH (Formula IIIa);

NH₂—(R²)—(X¹)—(Zₙ)—(X²)—(R¹)—COOH (Formula IIIb);

wherein
“R¹” is an alpha subunit from any multi-subunit first cytokine, preferably either IL-12-alpha subunit (p35) or IL-27 alpha subunit (p28), more preferably a subunit of SEQ ID No: 45 or 47
“R²” is a beta subunit from any multi-subunit first cytokine, preferably either IL-12-beta subunit (p40) or IL-27 beta subunit (EBI3), more preferably a subunit of SEQ ID No: 46 or 48;
wherein when R¹ is an alpha subunit of the first cytokine, R² is a beta subunit of the first cytokine; or when R¹ is p35, R² is p40; or when R¹ is p28, R² is EBI3; or when R¹ is SEQ ID No: 45 or 47, R² is SEQ ID No: 46 or 48; or when R¹ is SEQ ID No: 46 or 48, R² is SEQ ID No: 45 or 47;
“X¹” is a VL or VH region obtained from a first monoclonal antibody; “X²” is a VH or VL region obtained from the first monoclonal antibody; wherein when X¹ is a VL, X² is a VH or when X¹ is a VH, X² is a VL;
“Z” is any cytokine that enhances the biological function of the multi-subunit cytokine, preferably IFNa-2a, IL-28, IL-29, and
“n” is an integer selected from 1-2,
wherein the first monoclonal antibody is an anti-ebola antibody that is engrafted with CDRs from a second antibody with specificity for VEGFR2, CD3, CD4, CD5, CD7, CD19, CD20, CD22, CD25, CD30, CD33, CD34, CD38, CD40, CD44, CD52, CD56, CD70, CD79B, CD117, CD123, CD138, CD147, IL-22R1, BCMA, CLL01, CD5, CD147, ILMP-1, SLAMF7, NY-ESO-1, TACI, CS-1, CXCR4, NKG2D, B7-H3, EGFR, PD-1, PDL-1, HER2, HER3, EpCAM, PSCA, MUC1, Lewis-Y, GPC3, AXL, Claudin18.2, GD2, CTLA-4, CEA, PDGFR, MESO, PSCA, PSMA, BCMA, or PSA.

In yet another aspect, the present disclosure relates to a to a method of using a diakine having two multi-subunit cytokines, such as IL12, IL-27, or IL-10, of formula (IV) in combination with a TAA targeting engineered immune cell or a BiTE, said diakine being Formula (IV):

NH₂—(R¹)-(Lₐ)-(X¹)-(Lₐ)-(W¹)-(L_{b})-(W²)-(Lₐ)-(X²)-(Lₐ)-(R²)—COOH (Formula IV);
wherein
“R¹” is an alpha subunit of a first cytokine, such as IL-12 or IL-27 or a first monomer of a homodimeric cytokine, such as IL-10, wherein R¹ is preferably p40;
“R²” is a beta alpha subunit of the first cytokine, such as IL-12 or IL-27 or a second monomer of the homodimeric cytokine, such as IL-10, wherein R² is preferably p35;
“Lₐ” is any linker; preferably SEQ ID No: 43-44;
“L_{b}” is any linker; preferably GGGSGGG or SEQ ID No.: 43;
“X¹” is a VL or VH region obtained from a first monoclonal antibody; “X²” is a VH or VL region obtained from the first monoclonal antibody; wherein when X¹ is a VL, X² is a VH or when X¹ is a VH, X² is a VL;
“W¹” is an alpha subunit of a first cytokine, such as IL-12 or IL-27 or a first monomer of a homodimeric cytokine, such as IL-10, preferably a first monomer of IL-10;
“W²” is a beta alpha subunit of the first cytokine, such as IL-12 or IL-27 or a second monomer of the homodimeric cytokine, such as IL-10, preferably a second monomer of IL-10,

wherein the first monoclonal antibody is engrafted with CDRs from an antibody with specificity for VEGFR2, CD3, CD4, CD5, CD7, CD19, CD20, CD22, CD25, CD30, CD33, CD34, CD38, CD40, CD44, CD52, CD56, CD70, CD79B, CD117, CD123, CD138, CD147, IL-22R1, BCMA, CLL01, CD5, CD147, ILMP-1, SLAMF7, NY-ESO-1, TACI, CS-1, CXCR4, NKG2D, B7-H3, EGFR, PD-1, PDL-1, HER2, HER3, EpCAM, PSCA, MUC1, Lewis-Y, GPC3, AXL, Claudin18.2, GD2, CTLA-4, CEA, PDGFR, MESO, PSCA, PSMA, BCMA, or PSA.

The above simplified summary of representative aspects serves to provide a basic understanding of the present disclosure. This summary is not an extensive overview of all contemplated aspects, and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplarily pointed out in the claims.

### BRIEF DESCRIPTION OF THE DRAWINGS

FIG. 1 is a schematic diagram of a single embodiment of a first generation IL-10 fusion protein, which is a cytokine fusion protein previously described in U.S. Pat. No. 10,858,412.

FIG. 2 is a schematic diagram of a diakine embodied in the present disclosure, wherein the dual cytokine fusion protein comprises terminally linked IL-10 monomers (or IL10 variants), where a second cytokine is incorporated into the linker between the VH and VL of a scFv.

FIG. 3 is a schematic diagram of a diakine embodied in the present disclosure, wherein the dual cytokine fusion protein comprises two multi-subunit cytokines, wherein one is terminally linked (e.g., IL-12 or IL-27) and another is fused between the linker region of the scFv (e.g., two IL-10 monomers or IL-10 variants thereof).

FIG. 4 is a schematic drawing of the proposed mechanism of utilizing a DK2¹⁰ engrafted with CDRs targeting, for example, VEGFR2.

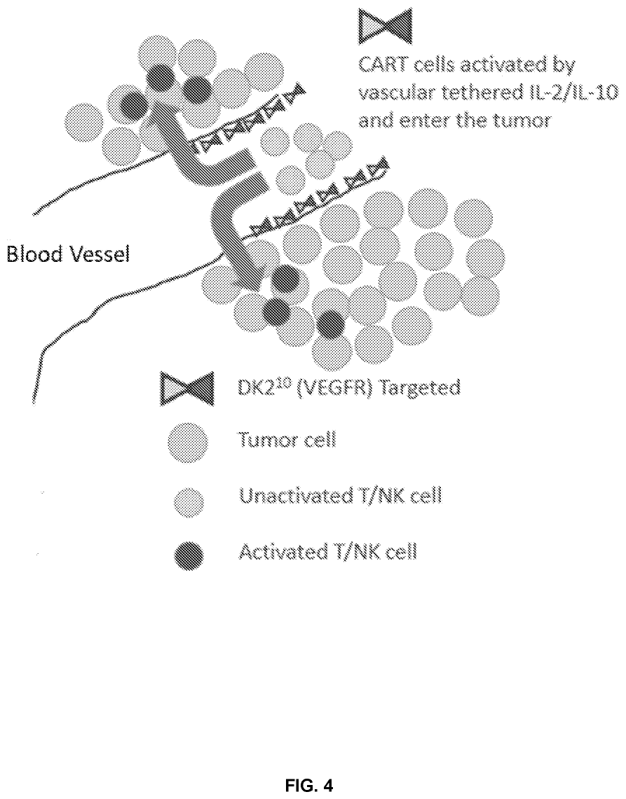

FIG. 5A-5F are grafts demonstrating the cytokine induction of IL-1, IFN-γ, TNF-α, IL-12p70, IFNα2a, and IL-6 in naïve PBMC from healthy donors in response to a diakine (DK2¹⁰), IL10, or IL2.

FIG. 6A-6D are grafts demonstrating the cytokine induction of IL-4, IL-17, IL-8, and GM-CSF in naïve PBMC from healthy donors in response to a diakine (DK2¹⁰), IL-10, or IL-2.

FIG. 7 is an evaluation of granzyme B levels in CD8+ T cells in response to escalating concentration of diakine in response to anti-CD3 stimulation at 24, 48, and 72 hours.

FIG. 8 is an evaluation of IFN-γ levels in CD8+ T cells in response to escalating concentration of diakine in response to anti-CD3 stimulation at 24, 48, and 72 hours.

FIG. 9 is an evaluation of TNF-α levels in CD8+ T cells in response to escalating concentration of diakine in response to anti-CD3 stimulation at 24, 48, and 72 hours.

FIGS. 10A & 10B shows the effects of combining a diakine (DK2¹⁰CD19) with a CAR T cell (CD20 CAR T) on reducing target tumor cells (Raji), wherein the CD8+ T cells are primed in the presence of a diakine for 1 day. FIG. 10A is a 3:1 effector to target ratio and FIG. 10B is a 1:3 effector to target ratio.

FIG. 11A-11F shows the effects of combining a diakine (DK2¹⁰egfr) with a BiTE (CD3×CD19 BiTE at 0.01 ng/mL)) on reducing target tumor cells (Raji), wherein the CD8+ T cells are primed in the presence of a diakine for 2 day. FIG. 11A-11D provides the cytokine secretion levels of TNF-alpha, IFN-gamma, granzyme B, and perforin of the CD8+ T cells in the presence of DK2¹⁰ and CD19 BiTE. FIGS. 11E and 11F provide the cytolytic profiles of CD8+ T cells when combined with DK2¹⁰ and CD19 BiTE.

FIG. 12A-12F shows the effects of combining a diakine (DK2¹⁰CD19) with a BiTE (CD3×CD20 BiTE at 0.1 ng/mL) on reducing target tumor cells (Raji), wherein the CD8+ T cells are primed in the presence of a diakine for 3 day. FIG. 12A-12D provides the cytokine secretion levels of TNF-alpha, IFN-gamma, granzyme B, and perforin of the CD8+ T cells in the presence of DK2¹⁰ and CD20 BiTE. FIGS. 12E and 12F provide the cytolytic profiles of CD8+ T cells when combined with DK2¹⁰ and CD20 BiTE.

FIG. 13 shows the effect of combining a diakine (DK7¹⁰) with a CD19 BiTE. The data provides a comparison of control (i.e., no DK7¹⁰, no CD19 BiTE), DK7¹⁰ alone, CD19 BiTE alone, and the combination of DK7¹⁰ and CD19 BiTE. The data shows that the combination of DK7¹⁰ and CD19 BiTE has enhanced cytolysis over CD19 BiTE.

FIG. 14 shows the effect of combining a diakine (DK12¹⁰) with a CD19 BiTE. The data provides a comparison of control (i.e., no DK12¹⁰, no CD19 BiTE), DK12¹⁰ alone, CD19 BiTE alone, and the combination of DK12¹⁰ and CD19 BiTE. The data shows that the combination of DK12¹⁰ and CD19 BiTE has enhanced cytolysis over CD19 BiTE.

### DETAILED DESCRIPTION

Exemplary aspects are described herein in the context of a class of dual cytokine fusion proteins termed diakines, whereby the diakines comprises IL-10, IL-12, or IL-27, and methods of treating cancer comprising administering a diakine comprising IL-10 and IL-2 (DK2¹⁰) or IL-10 and IL-7 (DK7¹⁰), IL-10 and IL-12 (DK12¹⁰), IL-10 and IL-15 (DK15¹⁰), IL-10 and IL-21 (DK21¹⁰), IL-10 and IFN-gamma (DKIFNa¹⁰) or IL-10 and IL-27 (DK27¹⁰) in combination with a tumor associated antigen (TAA) targeting engineered immune cells or ACT (such as a CAR T, TCT T, TIL or NK) or a BiTE. Those of ordinary skill in the art will understand that the following description is illustrative only and is not intended to be in any way limiting. Other aspects will readily suggest themselves to a person of skill in the art having the benefit of this disclosure. Reference will now be made in detail to implementations of the exemplary aspects as illustrated in the accompanying following disclosure and drawings. The same reference indicators will be used to the extent possible throughout the drawings and the following description to refer to the same or like items.

Although a number of methods and materials similar or equivalent to those described herein can be used in the practice of the various described embodiments, the preferred materials and methods are described herein.

Unless otherwise indicated, the embodiments described herein employ conventional methods and techniques of molecular biology, biochemistry, pharmacology, chemistry, and immunology, well known to a person skilled in the art. Many of the general techniques for designing and fabricating the IL-2, IL-7, IL-10, IL-12, IL-15, IL-21, IFN-alpha, or IL-27 variants, including but not limited to human, mouse, CMV and/or EBV forms of IL-10, as well as the assays for testing the IL-10 variants, aglycosylated or deglycosylated forms of each of the aforementioned cytokines and variants thereof, which are achieved by known methods readily available and detailed in the art. See, e.g., Sambrook, et al., Molecular Cloning: A Laboratory Manual (2nd Edition, 1989); Methods In Enzymology (S. Colowick and N. Kaplan eds., Academic Press, Inc.); Handbook of Experimental Immunology, Vols. I-IV (D. M. Weir and C. C. Blackwell eds., Blackwell Scientific Publications); A. L. Lehninger, Biochemistry (Worth Publishers, Inc., current addition). N-terminal aldehyde based PEGylation chemistry is also well known in the art.

### Definitions

The following terms will be used to describe the various embodiments discussed herein, and are intended to be defined as indicated below.

As used herein in describing the various embodiments, the singular forms “a”, “an” and “the” include plural referents unless the content clearly dictates otherwise.

The term “about”, refers to a deviance of between 0.0001-5% from the indicated number or range of numbers. In one embodiment, the term “about”, refers to a deviance of between 1-10% from the indicated number or range of numbers. In one embodiment, the term “about”, refers to a deviance of up to 25% from the indicated number or range of numbers. In a more specific embodiment, the term “about” refers to a difference of 1-25% in terms of nucleotide sequence homology or amino acid sequence homology when compared to a wild-type sequence.

The term “interleukin-10” or “IL-10” refers to a protein comprising two identical subunits non-covalently joined to form a homodimer, where IL-10 is an intercalated dimer of two six helix bundle (helix A-F). As used herein, unless otherwise indicated “interleukin-10” and “IL-10” refers to human IL-10 (“hIL-10”; Genbank Accession No. NP_000563; or U.S. Pat. No. 6,217,857) protein (SEQ ID No: 1) or nucleic acid (SEQ ID No: 2); mouse IL-10 (“mIL-10”; Genbank Accession No: M37897; or U.S. Pat. No. 6,217,857) protein (SEQ ID No: 7) or nucleic acid (SEQ ID No: 8); or viral IL-10, (“vIL-10”). Viral IL-10 homologs may be derived from EBV or CMV (Genbank Accession Nos. NC_007605 and DQ367962, respectively). The term EBV-IL10 refers to the EBV homolog of IL-10 protein (SEQ ID No: 3) or the nucleic acid (SEQ ID No: 4). The term CMV-IL10 refers to the CMV homolog of IL-10 protein (SEQ ID No: 5) or the nucleic acid (SEQ ID No: 6). The term “monomeric” or “monomer of” IL-10, as used herein, refers to the individual subunits of IL-10 or variant IL-10 that, when non-covalently joined, form a homodimer of IL-10 or variant IL-10. The terms “wild-type,” “wt” and “native” are used interchangeably herein to refer to the sequence of the protein (e.g. IL-10, CMV-IL10 or EBV IL-10) as commonly found in nature in the species of origin of the specific IL-10 in question. For example, the term “wild-type” or “native” EBV IL-10 would thus correspond to an amino acid sequence that is most commonly found in nature.

The term “interleukin-12” or “IL-12” refers to a protein comprising an alpha (p35) and beta (p40) subunit, non-covalently joined to form a heterodimer. As used herein, unless otherwise indicated “interleukin-12” and “IL-12” refers to human, mouse, or variant forms thereof. For example, the term “wild-type” or “native” would thus correspond to an amino acid sequence that is most commonly found in nature for the alpha and beta subunits.

The term “interleukin-27” or “IL-27” refers to a protein comprising an alpha (p28) and beta (EBI3) subunit, non-covalently joined to form a heterodimer. As used herein, unless otherwise indicated “interleukin-27” and “IL-27” refers to human; mouse, or variant forms thereof. For example, the term “wild-type” or “native” would thus correspond to an amino acid sequence that is most commonly found in nature for the alpha and beta subunits.

The terms “variant,” “analog” and “mutein” refer to biologically active derivatives of the reference molecule, that retain a desired activity, such as, for example, anti-inflammatory activity. Generally, the terms “variant,” “variants,” “analog” and “mutein” as it relates to a polypeptide refers to a compound or compounds having a native polypeptide sequence and structure with one or more amino acid additions, substitutions (which may be conservative in nature), and/or deletions, relative to the native molecule. As such, the terms “IL-10 variant”, “variant IL-10,” “IL-10 variant molecule,” and grammatical variations and plural forms thereof are all intended to be equivalent terms that refer to an IL-10 amino acid (or nucleic acid) sequence that differs from wild-type IL-10 anywhere from 1-25% in sequence identity or homology. Thus, for example, an EBV IL-10 variant molecule is one that differs from wild-type EBV IL-10 by having one or more amino acid (or nucleotide sequence encoding the amino acid) additions, substitutions and/or deletions. Thus in one form, an EBV IL-10 variant is one that differs from the wild type sequence of SEQ ID No.:3 by having about 1% to 25% difference in sequence homology, which amounts to about 1-42 amino acid difference. In one embodiment, an IL-10 variant is an EBV IL-10 comprising a A75I amino acid mutation (internally denoted as “DV06”; SEQ ID No: 14), or both V31L and a A75I amino acid mutations (internally denoted as “DV07”; SEQ ID No: 16). The terms “IL-12 variant”, “variant IL-12,” “IL-12 variant molecule,” “IL-27 variant” “variant IL-27,” “IL-27 variant molecule,” and grammatical variations and plural forms thereof are all intended to be equivalent terms that refer to an IL-12 or IL-27 amino acid (or nucleic acid) sequence that differs from wild-type IL-12 or IL-27. The difference in amino acid sequence for IL-12 or IL-27 may be additions, deletions, or substitutions within the alpha, beta, or both subunits such that there is anywhere from 1-25% in sequence identity or homology. These variant forms include modifications to the glycosylation (deglycosylated or aglycosylated) forms thereof to the protein.

The term “fusion protein” refers to a combination or conjugation of two or more proteins or polypeptides that results in a novel arrangement of proteins that do not normally exist naturally. The fusion protein is a result of covalent linkages of the two or more proteins or polypeptides. The two or more proteins that make up the fusion protein may be arranged in any configuration from amino-terminal end (“NH₂”) to carboxy-terminal end (“COOH”). Thus, for example, the carboxy-terminal end of one protein may be covalently linked to either the carboxy terminal end or the amino terminal end of another protein. Exemplary fusion proteins may include combining a monomeric IL-10 or a monomeric variant IL-10 molecule with one or more antibody variable domains (i.e., VH and/or VL) or single chain variable region (“scFv”).

The term “homolog,” “homology,” “homologous” or “substantially homologous” refers to the percent identity between at least two polynucleotide sequences or at least two polypeptide sequences. Sequences are homologous to each other when the sequences exhibit at least about 50%, preferably at least about 75%, more preferably at least about 80%-85%, preferably at least about 90%, and most preferably at least about 95%-98% sequence identity over a defined length of the molecules.

The term “sequence identity” refers to an exact nucleotide-by-nucleotide or amino acid-by-amino acid correspondence. The sequence identity may range from 100% sequence identity to 50% sequence identity. A percent sequence identity can be determined using a variety of methods including but not limited to a direct comparison of the sequence information between two molecules (the reference sequence and a sequence with unknown percent identity to the reference sequence) by aligning the sequences, counting the exact number of matches between the two aligned sequences, dividing by the length of the reference sequence, and multiplying the result by 100. Readily available computer programs can be used to aid in the identification of percent identity.

The terms “subject,” “individual” or “patient” are used interchangeably herein and refer to a vertebrate, preferably a mammal. Mammals include, but are not limited to, murine, rodent, simian, human, farm animals, sport animals, and certain pets.

The term “administering” includes routes of administration which allow the active ingredient of the application to perform their intended function.

A “therapeutically effective amount” or “effective amount” as it relates to, for example, administering the EBV IL-10 variants, fusion proteins, dual cytokine fusion proteins, or DK2¹⁰, DK7¹⁰, DK1210, DK1510 DK21¹⁰, DK27¹⁰, or DKIFNa¹⁰ thereof described herein, refers to an amount sufficient to promote certain biological activities, especially in the context of combining with TAA targeting engineered immune cells or ACT or BiTES. These might include, for example, suppression of myeloid cell function, enhanced Kupffer cell activity, and/or lack of any effect on CD8⁺ T cells or enhanced CD8⁺ T-cell activity as well as blockade of mast cell upregulation of Fc receptor or prevention of degranulation or to promote or enhance to effects of combination therapeutics (e.g., CAR-T therapies, or BiTES). Thus, an “effective amount” will treat, ameliorate or prevent a symptom or sign of the medical condition. Effective amount also means an amount sufficient to allow or facilitate diagnosis.

The term “treat” or “treatment” refers to a method of reducing the effects of a disease or condition. Treatment can also refer to a method of reducing the underlying cause of the disease or condition itself rather than just the symptoms. The treatment can be any reduction from native levels and can be, but is not limited to, the complete ablation of the disease, condition, or the symptoms of the disease or condition.

The term “diakine” or “DK”, as used in this application, refers to a general class of dual cytokine fusion protein comprising IL-10 (monomers of IL-10), IL-12, or IL-27 or variants thereof fused together with another cytokine onto a half-life extending antigen targeting domain. Specifically, a diakine takes the form of Formula I, II, IIIa, IIIb, IV.

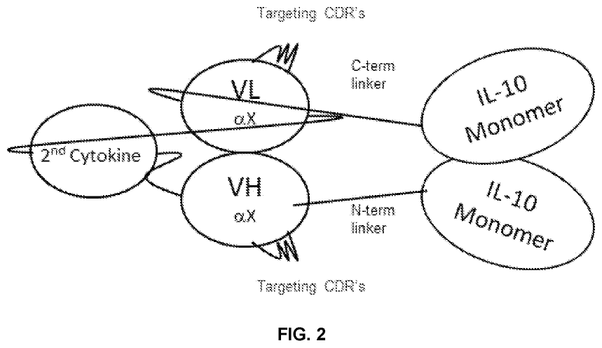

The term “DK2¹⁰” refers to a diakine, which is schematically represented in FIG. 2, comprising an IL-10 or IL-10 variant (e.g., SEQ ID No. 10), an IL-2 (e.g., SEQ ID No: 9) that is linked between the VH and VL regions of a scFv, where the diakine may be targeted or untargeted. Targeted DK2¹⁰ may be made into a targeting molecule by using a scFv that binds to a specific antigen, such as a TAA, or engrafting onto a scFv scaffolding CDRs from an antibody that target the TAA. These molecules will be denoted as DK2¹⁰(target name). For example, DK2¹⁰ that is targeted to EGFR will be denoted as “DK2¹⁰(egfr)” or “DK2¹⁰egfr.” In one embodiment, DK2¹⁰egfr is SEQ ID No: 19. In another example, DK2¹⁰ that is targeted to HER2 will be denoted as “DK2¹⁰(her2)” or “DK2¹⁰her2.” In one embodiment, DK2¹⁰her2 is SEQ ID No: 21, 23, or 25.

The term “DK7¹⁰” refers to a diakine, which is schematically represented in FIG. 2, comprising an IL-7 or an IL-7 variant (such as an aglycosylated or deglycosylated form) and an IL-10 and IL-10 variant (e.g., SEQ ID No: 10, or aglycosylated or deglycosylated forms thereof) that is linked between the VH and VL regions of a scFv, where the diakine may be targeted or untargeted. Targeted DK7¹⁰ may be made into a targeting molecule by using a scFv that binds to a specific antigen, such as a TAA or engrafting onto a scFv scaffolding CDRs from an antibody that target the TAA. These molecules will be denoted as DK7¹⁰(target name). For example, DK7¹⁰ that is targeted to EGFR will be denoted as “DK7¹⁰(egfr)” or “DK7¹⁰egfr.” In one embodiment, DK7¹⁰egfr is SEQ ID No: 36. In another example, DK7¹⁰ that is targeted to: (a) HER2 will be denoted as “DK7¹⁰(her2)” or “DK12¹⁰her2”; and (b) CD20 will be denoted “DK7¹⁰(CD20)” or “DK12¹⁰CD20” In one embodiment, DK7¹⁰her2 is SEQ ID No: 37 and DK7¹⁰CD20 is SEQ ID No: 38.

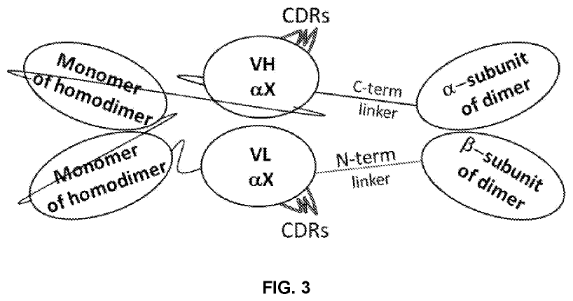

The term “DK12¹⁰” refers to a diakine, which is schematically represented in FIG. 3, comprising an IL-12 or an IL-12 variant (such as an aglycosylated or deglycosylated form) and an IL-10 and IL-10 variant (e.g., SEQ ID No: 10, or aglycosylated or deglycosylated forms thereof) that is linked between the VH and VL regions of a scFv, where the diakine may be targeted or untargeted. Targeted DK12¹⁰ may be made into a targeting molecule by using a scFv that binds to a specific antigen, such as a TAA or engrafting onto a scFv scaffolding CDRs from an antibody that target the TAA. These molecules will be denoted as DK12¹⁰(target name). For example, DK12¹⁰ that is targeted to EGFR will be denoted as “DK12¹⁰(egfr)” or “DK12¹⁰egfr.” In one embodiment, DK12¹⁰egfr is SEQ ID No: 26-32. In another example, DK12¹⁰ that is targeted to: (a) HER2 will be denoted as “DK12¹⁰(her2)” or “DK12¹⁰her2”; and (b) CD20 will be denoted “DK12¹⁰(CD20)” or “DK12¹⁰CD20” In one embodiment, DK12¹⁰CD20 is SEQ ID No: 34 or 35.

The term “DK15¹⁰” refers to a diakine, which is schematically represented in FIG. 2, comprising an IL-15 or an IL-15 variant (such as an aglycosylated or deglycosylated form) and an IL-10 and IL-10 variant (e.g., SEQ ID No: 10, or aglycosylated or deglycosylated forms thereof) that is linked between the VH and VL regions of a scFv, where the diakine may be targeted or untargeted. Targeted DK15¹⁰ may be made into a targeting molecule by using a scFv that binds to a specific antigen, such as a TAA or engrafting onto a scFv scaffolding CDRs from an antibody that target the TAA. These molecules will be denoted as DK15¹⁰(target name). For example, DK15¹⁰ that is targeted to EGFR will be denoted as “DK15¹⁰(egfr)” or “DK15¹⁰egfr.”

The term “DK21¹⁰” refers to a diakine, which is schematically represented in FIG. 2, comprising an IL-21 or an IL-21 variant (such as an aglycosylated or deglycosylated form) and an IL-10 and IL-10 variant (e.g., SEQ ID No: 10, or aglycosylated or deglycosylated forms thereof) that is linked between the VH and VL regions of a scFv, where the diakine may be targeted or untargeted. Targeted DK21¹⁰ may be made into a targeting molecule by using a scFv that binds to a specific antigen, such as a TAA or engrafting onto a scFv scaffolding CDRs from an antibody that target the TAA. These molecules will be denoted as DK21¹⁰(target name). For example, DK21¹⁰ that is targeted to EGFR will be denoted as “DK21¹⁰(egfr)” or “DK21¹⁰egfr.”

The term “DK27¹⁰” refers to a diakine, which is schematically represented in FIG. 2, comprising an IL-27 or an IL-27 variant (such as an aglycosylated or deglycosylated form) and an IL-10 and IL-10 variant (e.g., SEQ ID No: 10, or aglycosylated or deglycosylated forms thereof) that is linked between the VH and VL regions of a scFv, where the diakine may be targeted or untargeted. Targeted DK27¹⁰ may be made into a targeting molecule by using a scFv that binds to a specific antigen, such as a TAA or engrafting onto a scFv scaffolding CDRs from an antibody that target the TAA. These molecules will be denoted as DK27¹⁰(target name). For example, DK27¹⁰ that is targeted to EGFR will be denoted as “DK27¹⁰(egfr)” or “DK27¹⁰egfr.”

The term “DKIFNa¹⁰” refers to a diakine, which is schematically represented in FIG. 2, comprising an IFNa or an IFNa variant (such as an aglycosylated or deglycosylated form) and an IL-10 and IL-10 variant (e.g., SEQ ID No: 10, or aglycosylated or deglycosylated forms thereof) that is linked between the VH and VL regions of a scFv, where the diakine may be targeted or untargeted. Targeted DKIFNa¹⁰ may be made into a targeting molecule by using a scFv that binds to a specific antigen, such as a TAA or engrafting onto a scFv scaffolding CDRs from an antibody that target the TAA. These molecules will be denoted as DKIFNa¹⁰(target name). For example, DKIFNa¹⁰ that is targeted to EGFR will be denoted as “DKIFNa¹⁰(egfr)” or “DKIFNa¹⁰egfr.”

The term “DK12^{IFNa}” refers to a diakine, which is schematically represented in FIG. 2, comprising an IFNa or an IFNa variant (such as an aglycosylated or deglycosylated form) and an IL-12 and IL-12 variant (e.g., aglycosylated or deglycosylated forms thereof) that is linked between the VH and VL regions of a scFv, where the diakine may be targeted or untargeted. Targeted DK12^{IFNa} may be made into a targeting molecule by using a scFv that binds to a specific antigen, such as a TAA or engrafting onto a scFv scaffolding CDRs from an antibody that target the TAA. These molecules will be denoted as DK12^{IFNa}(target name). For example, DK12^{IFNa} that is targeted to EGFR will be denoted as “DK12^{IFNa} (egfr)” or “DK12^{IFNa} egfr.”

The term “tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

A tumor can be benign or malignant. A benign tumor is characterized as not undergoing metastasis. A malignant cell is a cancer cell and can undergo metastasis. Tumors on which the methods of the present application can be performed include, without limitation, adenoma, angio-sarcoma, astrocytoma, epithelial carcinoma, germinoma, glioblastoma, glioma, hamartoma, hemangioendothelioma, hemangiosarcoma, hematoma, hepato-blastoma, leukemia, lymphoma, medulloblastoma, melanoma, neuroblastoma, osteosarcoma, retinoblastoma, rhabdomyosarcoma, sarcoma, and teratoma. The tumor can be chosen from acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangio-carcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyo sarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic, papillary serous adeno-carcinoma, pineal cell, pituitary tumors, plasmacytoma, pseudo-sarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

Dual Cytokine Fusion Protein Structure

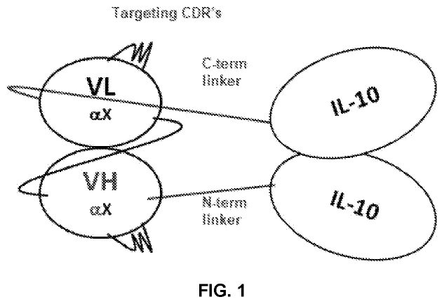

The present disclosure relates to the use of a diakine, which was previously described in U.S. application Ser. No. 17/199,239 (filed Mar. 11, 2021, and incorporated by reference in its entirety), to augment or enhance the function of known immunotherapies, such as TAA targeting engineered immune cells or BiTE therapy. Briefly, the diakine used in the method of the present application, which is an improvement on an earlier version of an IL-10 fusion protein previously described in U.S. Pat. Nos. 10,858,412 and 10,975,133 (incorporated by reference it its entirety), comprises two monomers of IL-10 or IL-10 variant molecules and a second cytokine molecule linked in the hinge region of a scFv. The diakine is built on the first-generation IL-10 fusion molecule (FIG. 1) which is described in U.S. Pat. Nos. 10,858,412 and 10,975,133. Briefly, the first-generation IL-10 fusion protein is constructed on a VH and VL scFv scaffolding featuring two monomers of IL-10 on each end (i.e., a first IL-10 monomer on the amino terminal end and a second IL-10 monomer on the carboxy terminal end). The scaffolding system comprises a scFv which was obtained from a human monoclonal anti-ebola antibody. with 6 complementarity-determining regions (“CDRs”) having CDRs 1-3 in the VH and CDRs 1-3 in the VL. The VH and VL regions are capable of targeting the IL-10 fusion protein to a specific antigen, which is accomplished by substituting the 6 CDR regions of the VH and VL pair (3 CDRs in the VH and 3 CDRs in the VL) with 6 CDR regions from a VH and VL of a receptor or antigen targeting antibody, or antigen binding fragment thereof. The ability to substitute and optimize the 6 CDR and framework regions of the scFv and to engraft CDRs into the scFv scaffolding described herein, is well known and practiced by those of skill in the art.

In a first aspect, the present application relates to a dual cytokine fusion protein, termed a diakine, comprising IL-10 or an IL-10 variant and at least one other cytokine, whereby the dual cytokine fusion protein has a combined or synergistic functionality when compared to the IL-10 fusion protein previously described in U.S. Pat. No. 10,858,412. FIG. 2 is a representative diagram of the diakine comprising IL-10. In particular, the diakine utilizes a scaffolding system made up of a scFv that comprises a VH and VL whereby two monomers of IL-10 terminate the dual cytokine fusion protein at the amino and carboxy terminal ends. The second cytokine is conjugated to the monomers of IL-10 (or IL-10 variants) by being fused between the VH and VL regions of the scFv, which is the hinge region of the scFv. The diakine is capable of forming a functional protein complex whereby the monomers of IL-10 (or IL-10 variants) homodimerize into a functional IL-10 molecule and the VH and VL regions form a pair that associate together to form a scFv complex that permits antigen binding and recognition.

In certain aspects, the diakine or dual cytokine fusion protein comprising IL-10 is a structure having formula I

NH₂-(IL-10)-(X¹)—(Zₙ)—(X²)—(IL-10)-COOH

wherein

“IL10” is a monomer of IL-10, wherein the IL-10 is human, mouse, CMV, or EBV IL-10, or a variant thereof, more preferably a IL10 is monomer comprising a sequence selected from SEQ ID Nos: 1, 3, 7, 9, or 10,

“X1” is a VL or VH region obtained from a first monoclonal antibody; “X2” is a VH or VL region obtained from the first monoclonal antibody; wherein when X1 is a VL, X2 is a VH or when X1 is a VH, X2 is a VL, preferably wherein the VH and VL regions are a scFv obtained from a human anti-ebola antibody, and the VH and VL regions are engrafted with 6 CDRs (3 from the VH and 3 from the VL) from a second antibody;

“Z” is any cytokine other than IL-10, preferably IL-6, IL-4, IL-1, IL-2, IL-3, IL-5, IL-7, IL-8, IL-9, IL-15, IL-21 IL-26, IL-27, IL-28, IL-29, GM-CSF, G-CSF, IFN-α, IFN-β, IFN-γ, TGF-β, or TNF-α, TNF-β, basic FGF, EGF, PDGF, IL-4, IL-11, or IL-13; and

“n” is an integer selected from 1-2.

In one preferred embodiment, the IL-10 is the high affinity variant termed DV07 that comprises substitutions at amino acid positions 31 and 75 of SEQ ID No: 10. In another embodiment, the VH and VL regions are a scFv obtained from any monoclonal antibody that is capable of binding to a TAA found on the surface of a solid or hematological tumor. In another aspect, the scFv is obtained from an antibody directed to VEGFR2. In one aspect, the scFv is obtained from a human monoclonal anti-Ebola antibody, wherein the 6 CDRs (3 in the VH and 3 in the VL) of the anti-Ebola antibody are optionally replaced with 6 CDRs from any monoclonal antibody that is capable of binding to a TAA. In another aspect, the scFv comprises the VH and VL framework regions from a human monoclonal anti-Ebola antibody that have been engrafted with CDRs having specificity for a TAA associated with solid or hematologic tumors. In one aspect, the VH and VL regions of the scFv are engrafted with 6 CDRs (3 from the VH and 3 from the VL) from an anti-VEGFR2, an antibody. In yet another preferred embodiment, the second cytokine is IL-2, more preferably SEQ ID No: 9.

In another aspect, the diakine or dual cytokine fusion protein comprising IL-10 is a structure having formula II

NH₂-(IL10)-(L)-(X¹)-(L)-(Zₙ)-(L)-(X²)-(L)-(IL10)-COOH

wherein

“IL-10” is an IL-10 monomer, such as but not limited to a human, mouse, CMV, EBV IL-10, or variants thereof;

“L” is a linker, preferably a linker of SEQ ID NO.: 39, 40, or 41;

“X¹” is a VL or VH region obtained from a first monoclonal antibody;

“X²” is a VH or VL region obtained from the first monoclonal antibody;

wherein when X¹ is a VL, X² is a VH or when X¹ is a VH, X² is a VL, and X¹ and X² together for a scFv;

“Z” is a second cytokine, wherein the second cytokine is a cytokine other than IL-10; and

“n” is an integer selected from 1-2.

In one preferred aspect, the IL-10 is the high affinity variant termed DV07 that comprises substitutions at amino acid positions 31 and 75 of SEQ ID No: 10. In another embodiment, the VH and VL regions are a scFv obtained from any monoclonal antibody that is capable of binding to a TAA found on the surface of a solid or hematological tumor. In another aspect, the scFv is obtained from an antibody directed to VEGFR2 In one aspect, the scFv is obtained from a human monoclonal anti-Ebola antibody, wherein the 6 CDRs (3 in the VH and 3 in the VL) of the anti-Ebola antibody are optionally replaced with 6 CDRs from any monoclonal antibody that is capable of binding to a TAA. In another aspect, the scFv comprises the VH and VL framework regions from a human monoclonal anti-Ebola antibody that have been engrafted with CDRs having specificity for a TAA associated with solid or hematologic tumors. In one aspect, the VH and VL regions of the scFv are engrafted with 6 CDRs (3 from the VH and 3 from the VL) from an anti-VEGFR2, ant antibody. In yet another preferred embodiment, the second cytokine is IL-2, more preferably SEQ ID No: 9.

In one embodiment, the IL-10 monomer includes any form of IL-10 including human (SEQ ID NO.:1), CMV (SEQ ID NO.: 5), EBV (SEQ ID NO.:3), or mouse (SEQ ID No: 7). In another embodiment, the IL-10 monomer is a modified or variant form of EBV IL-10 (SEQ ID NO.: 3), including those that are described in U.S. Pat. No. 10,858,412. In a preferred embodiment, the EBV IL-10 comprises two substitutions in SEQ ID No. 3 at amino acid position 31 and 75 (“DV07”). In yet another embodiment, the IL-10 monomer is a sequence of SEQ ID No: 1, 3, 7, or 10. The first and second monomers of IL-10 or IL-10 variant molecule are each located at the terminal ends of the fusion protein (i.e., the first monomer at the amino terminal end and the second monomer at the carboxy terminal end) as represented by FIG. 2.

In another aspect, the present disclosure relates to a method of using an IL-12 diakine of formula (III) in combination with a TAA targeting engineered immune cell or a BiTE

NH₂—(R¹)—(X¹)—(Zₙ)—(X²)—(R²)—COOH (Formula IIIa);

NH₂—(R²)—(X¹)—(Zₙ)—(X²)—(R¹)—COOH (Formula IIIb);

wherein
“R¹” is an alpha subunit from any multi-subunit first cytokine, preferably either IL-12-alpha subunit (p35) or IL-27 alpha subunit (p28), more preferably a subunit of SEQ ID No: 45 or 47;
“R²” is a beta subunit from any multi-subunit first cytokine, preferably either IL-12-beta subunit (p40) or IL-27 beta subunit (EBI3), more preferably a subunit of SEQ ID No: 46 or 48;
wherein when R¹ is an alpha subunit of the first cytokine, R² is a beta subunit of the first cytokine; or when R¹ is p35, R² is p40; or when R¹ is p28, R² is EBI3; or when R¹ is SEQ ID No: 45 or 47, R² is SEQ ID No: 46 or 48; or when R¹ is SEQ ID No: 46 or 48, R² is SEQ ID No: 45 or 47;
“X¹” is a VL or VH region obtained from a first monoclonal antibody; “X²” is a VH or VL region obtained from the first monoclonal antibody; wherein when X¹ is a VL, X² is a VH or when X¹ is a VH, X² is a VL;
“Z” is any cytokine that enhances the biological function of the multi-subunit cytokine, preferably IFNa-2a, IL-28, IL-29, and
“n” is an integer selected from 1-2,
wherein the first monoclonal antibody is an anti-ebola antibody that is engrafted with CDRs from a second antibody with specificity for VEGFR2, CD3, CD4, CD5, CD7, CD19, CD20, CD22, CD25, CD30, CD33, CD34, CD38, CD40, CD44, CD52, CD56, CD70, CD79B, CD117, CD123, CD138, CD147, IL-22R1, BCMA, CLL01, CD5, CD147, ILMP-1, SLAMF7, NY-ESO-1, TACI, CS-1, CXCR4, NKG2D, B7-H3, EGFR, PD-1, PDL-1, HER2, HER3, EpCAM, PSCA, MUC1, Lewis-Y, GPC3, AXL, Claudin18.2, GD2, CTLA-4, CEA, PDGFR, MESO, PSCA, PSMA, BCMA, or PSA.

In yet another aspect, the present disclosure relates to a to a method of using a diakine having two multi-subunit cytokines, such as IL12, IL-27, or IL-10, of formula (IV) in combination with a TAA targeting engineered immune cell or a BiTE, said diakine being Formula (IV):

NH₂—(R¹)-(Lₐ)-(X¹)-(Lₐ)-(W¹)-(L_{b})-(W²)-(Lₐ)-(X²)-(Lₐ)-(R²)—COOH (Formula IV);
wherein
“R¹” is an alpha subunit of a first cytokine, such as IL-12 or IL-27 or a first monomer of a homodimeric cytokine, such as IL-10, wherein R¹ is preferably p40;
“R²” is a beta alpha subunit of the first cytokine, such as IL-12 or IL-27 or a second monomer of the homodimeric cytokine, such as IL-10, wherein R² is preferably p35;
“Lₐ” is any linker; preferably SEQ ID No.: 43 or 44;
“L_{b}” is any linker; preferably SEQ ID No: GGGSGGG or SEQ ID No.: 42;
“X¹” is a VL or VH region obtained from a first monoclonal antibody; “X²” is a VH or VL region obtained from the first monoclonal antibody; wherein when X¹ is a VL, X² is a VH or when X¹ is a VH, X² is a VL;
“W¹” is an alpha subunit of a first cytokine, such as IL-12 or IL-27 or a first monomer of a homodimeric cytokine, such as IL-10, preferably a first monomer of IL-10;
“W²” is a beta alpha subunit of the first cytokine, such as IL-12 or IL-27 or a second monomer of the homodimeric cytokine, such as IL-10, preferably a second monomer of IL-10,

wherein the first monoclonal antibody is engrafted with CDRs from an antibody with specificity for VEGFR2, CD3, CD4, CD5, CD7, CD19, CD20, CD22, CD25, CD30, CD33, CD34, CD38, CD40, CD44, CD52, CD56, CD70, CD79B, CD117, CD123, CD138, CD147, IL-22R1, BCMA, CLL01, CD5, CD147, ILMP-1, SLAMF7, NY-ESO-1, TACI, CS-1, CXCR4, NKG2D, B7-H3, EGFR, PD-1, PDL-1, HER2, HER3, EpCAM, PSCA, MUC1, Lewis-Y, GPC3, AXL, Claudin18.2, GD2, CTLA-4, CEA, PDGFR, MESO, PSCA, PSMA, BCMA, or PSA.

The VH and VL regions are from an antibody, antibody fragment, or antigen binding fragment thereof. The antigen binding fragment includes, but is not limited to, a scFv, Fab, F(ab′)₂, V-NAR, diabody, or nanobody. Preferably the VH and VL, are from a single chain variable fragment (“scFv”). In an aspect, the scFv is obtained from a human monoclonal anti-Ebola antibody. In another aspect, the scFv comprises the framework region from an anti-Ebola antibody and 6 CDRs (3 VH and 3 VL) from a monoclonal antibody specific for any TAA that is expressed on the surface of a solid or hematological tumor. The scFv antibody or the engraftable CDRs are obtained from a monoclonal antibody selected from an antibody that is specific for VEGFR2, CD3, CD4, CD5, CD7, CD19, CD20, CD22, CD25, CD30, CD33, CD34, CD38, CD40, CD44, CD52, CD56, CD70, CD79B, CD117, CD123, CD138, CD147, IL-22R1, BCMA, CLL01, CD5, CD147, ILMP-1, SLAMF7, NY-ESO-1, TACI, CS-1, CXCR4, NKG2D, B7-H3, EGFR, PD-1, PDL-1, HER2, HER3, EpCAM, PSCA, MUC1, Lewis-Y, GPC3, AXL, Claudin18.2, GD2, CTLA-4, CEA, PDGFR, MESO, PSCA, PSMA, BCMA, or PSA, or multitargeting forms thereof.

In another aspect, the diakine comprising IL-10, IL-12, or IL-27 includes a VH and VL pair from a single antibody. The VH and VL pair act as a scaffolding onto which monomers of IL-10 or variants thereof may be attached such that the monomers of IL-10 or variants thereof may be able to homodimerize into a functioning IL-10 molecule. A person of skill in the art will therefore appreciate that the VH and VL scaffolding used in the fusion protein may be selected based on the desired physical attributes needed for proper homodimerization of the IL-10 monomers or IL-10 monomer variants and/or the desire to maintain VH and VL targeting ability. Likewise, a person of skill will also understand that the 6 CDRs within the VH and VL pair (3 CDRs from the VH and 3 CDRs from VL) may also be substituted with 6 CDRs from other antibodies to obtain a specifically targeted fusion protein. In one embodiment, 3 CDRs from a VH and 3 CDRs from a VL (i.e., a VH and VL pair) of any monoclonal antibody may be engrafted into a scaffolding system comprising SEQ No: 12, or 15. The scaffolding system described in SEQ ID No: 12 or 15, when fabricated as a diakine, will also include a second cytokine linked within the hinge region of the VH and VL portions of the molecule. It is also envisioned that if the dual cytokine fusion protein is not intended to target any specific antigen, a VH and VL pair may be selected as the scaffolding that does not target any particular antigen (or is an antigen in low abundance in vivo), such as the VH and VL pair from an anti-HIV and/or anti-Ebola antibody. Thus, in an embodiment, the IL-10 fusion protein of the present application may include a VH and VL pair from a human anti-Ebola antibody, more preferably the VH and VL sequence found in SEQ ID No: 12 or 15. The fusion protein may comprise a range of 1-4 variable regions. In another embodiment, the variable regions may be from the same antibody or from at least two different antibodies.

In another aspect, the target specificity of the antibody variable chains or VH and VL pair or the 6 CDRs of the VH and VL pair may include, but not limited to those targeting proteins, cellular receptors, and/or tumor associated antigens. In another embodiment, the CDR regions from any VH and VL pair may be engrafted into the scaffolding system described above, such scaffolding preferably includes a system termed Debo (schematically represented by FIG. 1), whereby IL-10 monomers are linked to a scFv comprising VH and VL regions of a human anti-Ebola antibody and the second cytokine is linked in the hinge region of the scFv (schematically represented by FIG. 2). More preferably engraftment into the Debo scaffolding system occurs in a scaffolding comprising a sequence of SEQ ID No: 12 or 15. In yet another embodiment, the variable regions or VH and VL pair or the 6 CDRs of the VH and VL pair are obtained from antibodies that target antigens associated with various diseases (e.g., cancer) or those that are not typically found or rarely found in the serum of a healthy subject, for example variable regions from antibodies directed to EGFR, PDGFR, VEGFR1, VEGFR2, Her2Neu, FGFR, GPC3, or other tumor associated antigens, MAdCAM, ICAM, VCAM, CD14 or other inflammation associated cell surface proteins, HIV and/or Ebola. Thus, in one embodiment, the variable regions are obtained or derived from anti-EGFR, anti-MAdCAM, anti-HIV (Chan et al, J. Virol, 2018, 92(18):e006411-19), anti-ICAM, anti-VCAM, anti-CD14, or anti-Ebola (US Published Application 2018/0180614, incorporated by reference in its entirety, especially mAbs described in Tables 2, 3, and 4) antibodies, for example. In another embodiment, the variable regions are obtained or derived from antibodies capable of enriching the concentration of cytokines, such as IL-10 and IL-2, to a specific target area so as to enable IL-10 and IL-2 to elicit its biological effect. Such an antibody might include those that target overexpressed or upregulated receptors or antigens in certain diseased regions or those that are specifically expressed in certain impacted areas. For example, the variable regions might be obtained from antibodies specific for epidermal growth factor receptor (EGFR); CD52; CD14; various immune check point targets, such as but not limited to PD-L1, PD-1, TIM3, BTLA, LAG3 or CTLA4; CD20; CD47; GD-2; VEGFR1; VEGFR2; HER2; PDGFR; EpCAM; ICAM (ICAM-1, -2, -3,-4, -5), VCAM, FAPα; 5T4; Trop2; EDB-FN; TGFβ Trap; MAdCAM, β7 integrin subunit; α4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; and SR-J1 to name a few. Other variable regions might include those that are obtained from antibodies specific for CD3, CD4, CD5, CD7, CD19, CD22, CD25, CD30, CD33, CD34, CD38, CD40, CD56, CD70, CD79B, CD117, CD123, CD138, CD147, B cell Maturation Antigen (BCMA), -type lectin like molecule-1 (CLL01), latent membrane protein 1 (LMP-1), signaling lymphatic activation molecule F7 (SLAMF7), NY-ESO-1, transmembrane activator and CAML interactor (TACI), CS-1, CXCR4, NKG2D, B7-H3, EGFR, HER3, EpCAM, mesothelin, PSCA, MUC1, Lewis-Y, GPC3, AXL, Claudin18.2, GD2, CTLA-4, CEA, PDGFR, mesothelin (MESO), PSCA, PSA. A monomer of IL-10 (e.g., human, CMV, or EBV) or variant IL-10 molecule (described herein) is conjugated to either the amino terminal end or the carboxy terminal end of a variable region (VH or VL), such that the monomer IL-10 or variant IL-10 molecule is able to dimerize with one another. In a preferred embodiment, the monomers of IL-10 (or variant IL-10) are fused to the VH and VL pair in accordance to formula I or II, wherein the IL-10 monomer is an EBV IL-10, DV05, DV06, or DV07 form of IL-10.

The diakine, dual cytokine fusion protein or dual cytokine fusion protein complex may also have an antigen targeting functionality. The diakine or dual cytokine fusion protein or dual cytokine fusion protein complex will comprise a VH and VL pair that is able to associate together to form an antigen binding site or ABS. In some configurations, the IL-10 monomers or IL-10 variant monomers thereof will be covalently linked to the end comprising the antigen binding site. The variable regions may be further modified (e.g., by addition, subtraction, or substitution) by altering one or more amino acids that reduce antigenicity in a subject. Other modifications to the variable region may include amino acids substitutions, deletions, or additions that are found outside of the 6 CDR regions of the VH and VL regions and serve to increase stability and expression of the VH and VL regions of the scFv. For example, the modifications may include modifications wherein the CDR regions are obtained from the VH and VL regions of an anti-EGFR or anti-VEGFR1 or anti-VEGFR2 antibody and the regions outside of the CDRs are optimized to stabilize the scFv and/or optimized to increase expression, which may be used as a basis for linking the second cytokine between the VH and VL regions of the scFv. To demonstrate that these types of modifications are within the purview of a skilled artisan, similar modifications to the CDR regions and regions outside of the CDRs were made to a molecule in DK2¹⁰ form comprising DV07 and targeting human HER2 (i.e., DK2¹⁰her2), such as those described in SEQ ID No: 52-54, or 55, more preferably SEQ ID No: 21 (variant 4) or 23 (variant 5). These and other modifications may also be made to a molecule in DK2¹⁰ form comprising DV07 and targeting human VEGFR1 or VEGFR2A and a person of skill in the art would be capable of determining other modifications that stabilize the scFv and/or to optimize the sequence for purposes of expression.

The VH and VL pair form a scaffolding onto which CDR regions obtained for a plurality of antibodies may be grafted or engrafted. Such antibody CDR regions include those antibodies known and described above. The CDR regions in the above described VH and VL scaffolding will include the following number of amino acid positions available for CDR engraftment/insertion:

| | |
|---|---|
| Heavy chain CDR1 | 3-7 amino acids |
| Heavy chain CDR2 | 7-11 amino acids |
| Heavy chain CDR3 | 7-11 amino acids |
| Light chain CDR1 | 9-14 amino acids |
| Light chain CDR2 | 5-9 amino acids |
| Light chain CDR3 | 7-11 amino acids |

In a preferred embodiment, the dual cytokine fusion protein comprising IL-10 will include the previously described scaffolding IL-10 fusion protein where the VH and VL pair is derived from an anti-ebola antibody (such as those described in SEQ ID No: 19) whereby the 6 CDR regions from the anti-ebola antibody are removed and engrafted with a VH and VL pair of a specific targeting antibody, such as but not limited to EGFR; CD52; CD14; various immune check point targets, such as but not limited to PD-L1, PD-1, TIM3, BTLA, LAG3 or CTLA4; CD19, CD20; CD22, CD47; GD-2; VEGFR1; VEGFR2; HER2; PDGFR; EpCAM; ICAM (ICAM-1, -2, -3, -4, -5), VCAM, CD14, FAPα; 5T4; Trop2; EDB-FN; TGFβ Trap; MAdCam, β7 integrin subunit; α4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; and SR-J1. In an embodiment, the 6 anti-ebola CDR regions are substituted with 6 CDR regions from anti-EGFR, anti-MAdCAM, anti-VEGFR1, anti-VEGFR2, anti-PDGFR, or anti-CD14, anti-CD19, anti-CD20, anti-CD22, more preferably an anti-VEGFR2 antibody.

In yet another aspect, the second cytokine, is fused between the VH and VL of a scFv, as depicted in FIG. 2. The second cytokine is conjugated, fused or linked between the VH or VL region of the scFv such that the second cytokine retains its functional properties. In one embodiment, the second cytokine is different from the IL-10 monomer. In another embodiment, the second cytokine is IL-10. In one embodiment, the second cytokine is IL-6, IL-4, IL-1, IL-2, IL-3, IL-5, IL-7, IL-8, IL-9, IL-15, IL-17, IL-21, IL-26, IL-27, IL-28a, IL28b, IL-29, TSLP, GM-CSF, G-CSF, interferons-α, -β, -γ, TGF-β, or tumor necrosis factors-α, -β, basic FGF, EGF, PDGF, IL-4, IL-11, or IL-13, or variants or muteins thereof, such as but not limited to high, medium and low receptor affinity variants. In a preferred embodiment, the second cytokine in the diakine comprising IL-10, is an IL-2. In a more preferred embodiment, the diakine or dual cytokine fusion protein is in DK2¹⁰ form, where the IL-10 monomer is DV07; the IL-10 variant molecule linked to a scaffolding system comprising the VH and VL framework regions from a human anti-Ebola antibody (i.e., Debo), and further comprising engraftment with CDRs obtained from an antibody selected from an anti-EGFR, anti-HER2, anti-CD14, anti-VEGFR1, anti-VEGFR2, anti-MAdCAM, or anti-PDGFR, anti-Cd19, anti-Cd20, anti-CD22, anti-CD3, anti-CD4, anti-CD5, anti-CD7, anti-CD25, anti-CD30, anti-CD33, anti-CD34, anti-CD38, anti-CD40, anti-CD52, anti-CD56, anti-CD70, anti-CD79B, anti-CD117, anti-CD123, anti-CD138, anti-CD147, anti-B cell maturation antigen (BCMA), anti-C-type lectin-like molecule-1 (CLL01), anti-CD5, anti-CD147, anti-latent membrane protein 1 (LMP-1), anti-signaling lymphocytic activation molecule F7 (SLAMF7), anti-NY-ESO-1, anti-transmembrane activator and CAML interactor (TACI), anti-CS-1, anti-CXCR4, anti-NKG2D, anti-B7-H3, anti-PD-1, anti-PDL-1, anti-HER3, anti-EpCAM, anti-mesothelin, anti-PSCA, anti-MUC1, anti-Lewis-Y, anti-GPC3, anti-AXL, anti-Claudini8.2, anti-GD2, anti-CTLA-4, anti-CEA, anti-mesothelin (MESO), anti-PSCA, or anti-PSA and a second cytokine selected from IFNα, IL2, IL7, IL15, IL21, IL28, IL29, or high, medium, or low affinity variants thereof, that is linked in the hinge region of the VH and VL pair. In a most preferred embodiment, the diakine or dual cytokine fusion protein is a fusion protein in DK2¹⁰ form comprising DV07 and engrafted with 6 CDRs from an anti-VEGFR2 antibody.

In another aspect, the diakine is a combination of a first cytokine (or high affinity variants thereof), a second cytokine (or high affinity variants thereof), and a targeting scFv (or CDRs obtained from monoclonal antibodies and engrafted into a framework region obtained from an anti-ebola antibody) as set forth in the Table 1 below:

TABLE 1

| 1^{ST} | 2^{nd} | Antibodies, scFv or CDRs derived |
|---|---|---|
| Cytokine | Cytokine | from antibodies specific for |
| IL10 | IL2 | VEGFR2 |
| IL10 | IL2 | Ebola |
| IL10 | IFNα, IL2, IL7, | CD19 |
| | IL15, IL21, IL28, | CD20 |
| | IL29 | CD22 |
| IL10 | IFNα, IL2, IL7, | PDGFR |
| | IL15, IL21, IL28, | |
| | IL29 | |
| IL10 | IFNα, IL2, IL7, | CD3, CD4, CD5, CD7, CD25, CD30, |
| | IL15, IL21, IL28, | CD33, CD34, CD38, CD40, CD52, |
| | IL29 | CD56, CD70, CD79B, CD117, CD123, |
| | | CD138, CD147, B cell maturation |
| | | antigen (BCMA), C-type lectin-like |
| | | molecule-1 (CLL01), CD5, CD147, |
| | | latent membrane protein 1 (LMP-1), |
| | | signaling lymphocytic activation |
| | | molecule F7 (SLAMF7), NY-ESO-1, |
| | | transmembrane activator and CAML |
| | | interactor (TACI), CS-1, CXCR4, |
| | | NKG2D, B7-H3, EGFR, PD-1, PDL-1, |
| | | HER2, HER3, EpCAM, mesothelin, |
| | | PSCA, MUC1, Lewis-Y, GPC3, AXL, |
| | | Claudin18.2, GD2, CTLA-4, CEA, |
| | | mesothelin (MESO), PSCA, or PSA |
| IL12 | IL-10, IL-28, | VEGFR2, Ebola, CD19, CD20, CD22, |
| | IL-29, IFNa | PDGFR, CD3, CD4, CD5, CD7, CD25, |
| | | CD30, CD33, CD34, CD38, CD40, |
| | | CD52, CD56, CD70, CD79B, CD117, |
| | | CD123, CD138, CD147, B cell |
| | | maturation antigen (BCMA), C-type |
| | | lectin-like molecule-1 (CLL01), |
| | | CD5, CD147, latent membrane |
| | | protein 1 (LMP-1), signaling |
| | | lymphocytic activation molecule |
| | | F7 (SLAMF7), NY-ESO-1, trans- |
| | | membrane activator and CAML |
| | | interactor (TACI), CS-1, CXCR4, |
| | | NKG2D, B7-H3, EGFR, PD-1, PDL-1, |
| | | HER2, HER3, EpCAM, mesothelin, |
| | | PSCA, MUC1, Lewis-Y, GPC3, AXL, |
| | | Claudin18.2, GD2, CTLA-4, CEA, |
| | | mesothelin (MESO), PSCA, or PSA |
| IL-27 | IL-10, IL-28, | VEGFR2, Ebola, CD19, CD20, CD22, |
| | IL-29, IFNa | PDGFR, CD3, CD4, CD5, CD7, CD25, |
| | | CD30, CD33, CD34, CD38, CD40, |
| | | CD52, CD56, CD70, CD79B, CD117, |
| | | CD123, CD138, CD147, BCMA, CLL01, |
| | | CD5, CD147, LMP-1, SLAMF7, NY- |
| | | ESO-1, TACI, CS-1, CXCR4, NKG2D, |
| | | B7-H3, EGFR, PD-1, PDL-1, HER2, |
| | | HER3, EpCAM, mesothelin, PSCA, |
| | | MUC1, Lewis-Y, GPC3, AXL, |
| | | Claudin18.2, GD2, CTLA-4, CEA, |
| | | mesothelin (MESO), PSCA, |
| | | or PSA |

In other aspects, a possible diakine or a method of using a combination of at least one of the diakines and a TAA targeted engineered immune cells, ACT, or BiTEs combinations include one of the following listed in Table 2a-2e).

TABLE 2a

| First Cytokine | scFv | Second Cytokine |
|---|---|---|
| hIL10 | CD19 | IL2 |
| (SEQ ID No. 1) | CD20 | (SEQ ID No. 9) |
| | CD22 | |
| | CD3 | |
| | CD38 | |
| | CD44 | |
| | BCMA | |
| | HER3 | |
| | PSCA | |
| | PSMA | |
| | PSA | |
| | GPC3 | |
| | IL22R | |
| | CD70 | |
| | CD33 | |
| | CXCR4 | |
| | CEA | |
| DV07 | CD19 | |
| (SEQ ID No. 3) | CD20 | |
| | CD22 | |
| | CD3 | |
| | CD38 | |
| | CD44 | |
| | BCMA | |
| | HER3 | |
| | PSCA | |
| | PSMA | |
| | PSA | |
| | GPC3 | |
| | IL22R | |
| | CD70 | |
| | CD33 | |
| | CXCR4 | |
| | CEA | |

TABLE 2b

| First Cytokine | scFv | Second Cytokine |
|---|---|---|
| hIL10 | CD19 | IL7 |
| (SEQ ID No. 1) | CD20 | (SEQ ID No. 50) |
| | CD22 | |
| | CD3 | |
| | CD38 | |
| | CD44 | |
| | BCMA | |
| | HER3 | |
| | PSCA | |
| | PSMA | |
| | PSA | |
| | GPC3 | |
| | IL22R | |
| | CD70 | |
| | CD33 | |
| | CXCR4 | |
| | CEA | |
| DV07 | CD19 | |
| (SEQ ID No. 3) | CD20 | |
| | CD22 | |
| | CD3 | |
| | CD38 | |
| | CD44 | |
| | BCMA | |
| | HER3 | |
| | PSCA | |
| | PSMA | |
| | PSA | |
| | GPC3 | |
| | IL22R | |
| | CD70 | |
| | CD33 | |
| | CXCR4 | |
| | CEA | |

TABLE 2c

| First Cytokine | scFv | Second Cytokine |
|---|---|---|
| hIL10 | CD19 | IL15 |
| (SEQ ID No. 1) | CD20 | (SEQ ID No. 51) |
| | CD22 | |
| | CD3 | |
| | CD38 | |
| | CD44 | |
| | BCMA | |
| | HER3 | |
| | PSCA | |
| | PSMA | |
| | PSA | |
| | GPC3 | |
| | IL22R | |
| | CD70 | |
| | CD33 | |
| | CXCR4 | |
| | CEA | |
| DV07 | CD19 | |
| (SEQ ID No. 3) | CD20 | |
| | CD22 | |
| | CD3 | |
| | CD38 | |
| | CD44 | |
| | BCMA | |
| | HER3 | |
| | PSCA | |
| | PSMA | |
| | PSA | |
| | GPC3 | |
| | IL22R | |
| | CD70 | |
| | CD33 | |
| | CXCR4 | |
| | CEA | |

TABLE 2d

| First Cytokine | scFv | Second Cytokine |
|---|---|---|
| hIL10 | CD19 | IFN-alpha |
| (SEQ ID No. 1) | CD20 | (SEQ ID No: 49) |
| | CD22 | |
| | CD3 | |
| | CD38 | |
| | CD44 | |
| | BCMA | |
| | HER3 | |
| | PSCA | |
| | PSMA | |
| | PSA | |
| | GPC3 | |
| | IL22R | |
| | CD70 | |
| | CD33 | |
| | CXCR4 | |
| | CEA | |
| DV07 | CD19 | |
| (SEQ ID No. 3) | CD20 | |
| | CD22 | |
| | CD3 | |
| | CD38 | |
| | CD44 | |
| | BCMA | |
| | HER3 | |
| | PSCA | |
| | PSMA | |
| | PSA | |
| | GPC3 | |
| | IL22R | |
| | CD70 | |
| | CD33 | |
| | CXCR4 | |
| | CEA | |

TABLE 2e

| First Cytokine | scFv | Second Cytokine |
|---|---|---|
| hIL10 | CD19 | IL21 |
| (SEQ ID No. 1) | CD20 | (SEQ ID No: 49) |
| | CD22 | |
| | CD3 | |
| | CD38 | |
| | CD44 | |
| | BCMA | |
| | HER3 | |
| | PSCA | |
| | PSMA | |
| | PSA | |
| | GPC3 | |
| | IL22R | |
| | CD70 | |
| | CD33 | |
| | CXCR4 | |
| | CEA | |
| DV07 | CD19 | |
| (SEQ ID No. 3) | CD20 | |
| | CD22 | |
| | CD3 | |
| | CD38 | |
| | CD44 | |
| | BCMA | |
| | HER3 | |
| | PSCA | |
| | PSMA | |
| | PSA | |
| | GPC3 | |
| | IL22R | |
| | CD70 | |
| | CD33 | |
| | CXCR4 | |
| | CEA | |

In still other aspects, the diakine or dual cytokine fusion protein comprising IL-10, IL-12 or IL-27 incorporates linkers. A person of skill in the art knows that linkers or spacers are used to achieve proper spatial configuration of the various fusion protein parts and therefore may select the appropriate linker to use in the formation of the dual cytokine fusion protein comprising IL-10. In a more preferred embodiment, the linker or spacer may be a random amino acid sequence (such as SEQ ID No: 39-44) or a constant region of an antibody. The constant region can be derived from, but not limited to IgG1, IgG2, IgG3, IgG4, IgA, IgM, IgD, or IgE. In one embodiment, the linker or spacer is a constant heavy (“CH”) region 1, CH₂, or CH₃. In another aspect, the linker or spacer may further comprise at least two interchain disulfide bonds.

In other aspects, the present disclosure relates to nucleic acid molecules that encode for the diakine or dual cytokine fusion protein comprising IL-10, IL-12, or IL-27 and a second cytokine. These nucleic acid molecules are described in U.S. application Ser. No. 17/110,104. The polynucleotide sequences that encode for the diakine or dual cytokine fusion protein comprising IL-10 and a second cytokine may also include modifications that do not alter the functional properties of the described dual cytokine fusion protein. Such modifications will employ conventional recombinant DNA techniques and methods. For example, the addition or substitution of specific amino acid sequences may be introduced into an IL-10 sequence at the nucleic acid (DNA) level using site-directed mutagenesis methods employing synthetic oligonucleotides, which methods are also well known in the art. In a preferred embodiment, the nucleic acid molecules encoding the dual cytokine fusion protein comprising IL-10 and a second cytokine may include insertions, deletions, or substitutions (e.g., degenerate code) that do not alter the functionality of the IL-10 variant molecule. The nucleotide sequences encoding the IL-10, IL-12, IL-27 variant and/or the overall fusion proteins described herein may differ from the amino acid sequences due to the degeneracy of the genetic code and may be 70-99%, preferably 70%, 75%, 80%, 85%, 90%, 95%, 96%, 97%, 98%, or 99%, homologous to the aforementioned sequences. Accordingly, an embodiment of the present disclosure includes a nucleic acid sequence that encodes a protein of SEQ ID Nos: 35, 46-58, or 59 but differing by 70-99% due to the degeneracy of the genetic code.

The nucleotide sequences encoding the diakine or dual cytokine fusion proteins described herein may further comprise well known sequences that aid in, for example, the expression, production, or secretion of the proteins. Such sequences may include, for example a leader sequence, signal peptide, and/or translation initiation sites/sequence (e.g. Kozak consensus sequence). The nucleotide sequences described herein may also include one of more restriction enzyme sites that allow for insertion into various expression systems/vectors.

In another aspect, the nucleotide sequences encoding the dual cytokine fusion protein may be used directly in gene therapy. In one embodiment, the variant IL-10, IL-12, or IL-27 molecules or fusion protein of the present application can be delivered by any method know in the art, including direct administration of the mutant IL-10, IL-12, or IL-27 proteins and gene therapy with a vector encoding the mutant IL-10 protein. Gene therapy may be accomplished using plasmid DNA or a viral vector, such as an adeno-associated virus vector, an adenovirus vector, a retroviral vector, etc. In some embodiments, the viral vectors of the application are administered as virus particles, and in others they are administered as plasmids (e.g. as “naked” DNA).

Other methods for the delivery of the nucleotide sequences include those which are already known in the art. These would include the delivery of the nucleotide sequences, such as but not limited to DNA, RNA, siRNA, mRNA, oligonucleotides, or variants thereof, encoding the IL-10 or IL-10 variant molecules by a cell penetrating peptide, a hydrophobic moiety, an electrostatic complex, a liposome, a ligand, a liposomal nanoparticle, a lipoprotein (preferably HDL or LDL), a folate targeted liposome, an antibody (such as Folate receptor, transferrin receptor), a targeting peptide, or by an aptamer. The nucleotide sequences encoding IL-10, IL-12 or IL-27 variant molecules may be delivered to a subject by direct injection, infusion, patches, bandages, mist or aerosol, or by thin film delivery. The nucleotide (or the protein) may be directed to any region that is desired for targeted delivery of a cytokine stimulus. These would include, for example, the lung, the GI tract, the skin, liver, brain though intracranial injection, deep seated metastatic tumor lesions via ultrasound guided injections.

In another aspect, the present disclosure relates to methods of preparing and purifying the diakine or dual cytokine fusion protein comprising IL-10, IL-12, or IL-27. For example, nucleic acid sequences that encode the diakine or dual cytokine fusion protein described herein may be used to recombinantly produce the fusion proteins. For example, using conventional molecular biology and protein expression techniques, the diakine or dual cytokine fusion protein described herein may be expressed and purified from mammalian cell systems. These systems include well known eukaryotic cell expression vector systems and host cells. A variety of suitable expression vectors may be used and are well known to a person skilled in the art, which can be used for expression and introduction of the variant IL-10, IL-12 or IL-27 molecules and fusion proteins. These vectors include, for example, pUC-type vectors, pBR-type vectors, pBI-type vectors, pGA-type, pBinI9, pBI121, pGreen series, pCAMBRIA series, pPZP series, pPCV001, pGA482, pCLD04541, pBIBAC series, pYLTAC series, pSB11, pSB1, pGPTV series, and viral vectors and the like can be used. Well known host cell systems include but not limited to expression in CHO cells.

The expression vectors harboring the diakine or dual cytokine fusion protein may also include other vector componentry required for vector functionality. For example, the vector may include signal sequences, tag sequences, protease identification sequences, selection markers and other sequences regulatory sequences, such as promoters, required for proper replication and expression of the dual cytokine fusion protein. The particular promoters utilized in the vector are not particularly limited as long as they can drive the expression of the dual cytokine fusion protein in a variety of host cell types. Likewise, the type of Tag promoters are not be limited as long as the Tag sequence makes for simplier or easier purification of expressed variant IL-10 molecule easier. These might include, for example, 6-histidine, GST, MBP, HAT, HN, S, TF, Trx, Nus, biotin, FLAG, myc, RCFP, GFP and the like can be used. Protease recognition sequences are not particularly limited, for instance, recognition sequences such as Factor Xa, Thrombin, HRV, 3C protease can be used. Selected markers are not particularly limited as long as these can detect transformed rice plant cells, for example, neomycin-resistant genes, kanamycin-resistant genes, hygromycin-resistant genes and the like can be used.

The diakine or dual cytokine fusion protein described above may also include additional amino acid sequences that aid in the recovery or purification of the fusion proteins during the manufacturing process. These may include various sequence modifications or affinity tags, such as but not limited to protein A, albumin-binding protein, alkaline phosphatase, FLAG epitope, galactose-binding protein, histidine tags, and any other tags that are well known in the art. See, e.g., Kimple et al (Curr. Protoc. Protein Sci., 2013, 73:Unit 9.9, Table 9.91, incorporated by reference in its entirety). In one aspect, the affinity tag is an histidine tag having an amino acid sequence of 6 histidines. The histidine tag may be removed or left intact from the final product. In another embodiment, the affinity tag is a protein A modification that is incorporated into the fusion protein (e.g., into the VH region of the fusion proteins described herein). A person of skill in the art will understand that any dual cytokine fusion protein sequence described herein can be modified to incorporate a protein A modification by inserting amino acid point substitutions within the antibody framework regions as described in the art.

In another aspect, the protein and nucleic acid molecules encoding dual cytokine fusion protein may be formulated as a pharmaceutical composition comprising a therapeutically effective amount of the dual cytokine fusion protein and a pharmaceutical carrier and/or pharmaceutically acceptable excipients. The pharmaceutical composition may be formulated with commonly used buffers, excipients, preservatives, stabilizers. The pharmaceutical compositions comprising the dual cytokine fusion protein is mixed with a pharmaceutically acceptable carrier or excipient. Various pharmaceutical carriers are known in the art and may be used in the pharmaceutical composition. For example, the carrier can be any compatible, non-toxic substance suitable for delivering the dual cytokine fusion protein compositions of the application to a patient. Examples of suitable carriers include normal saline, Ringer's solution, dextrose solution, and Hank's solution. Carriers may also include any poloxamers generally known to those of skill in the art, including, but not limited to, those having molecular weights of 2900 (L64), 3400 (P65), 4200 (P84), 4600 (P85), 11,400 (F88), 4950 (P103), 5900 (P104), 6500 (P105), 14,600 (F108), 5750 (P123), and 12,600 (F127). Carriers may also include emulsifiers, including, but not limited to, polysorbate 20, polysorbate 40, polysorbate 60, and polysorbate 80, to name a few. Non-aqueous carriers such as fixed oils and ethyl oleate may also be used. The carrier may also include additives such as substances that enhance isotonicity and chemical stability, e.g., buffers and preservatives, see, e.g., Remington's Pharmaceutical Sciences and U.S. Pharmacopeia: National Formulary, Mack Publishing Company, Easton, Pa. (1984). Formulations of therapeutic and diagnostic agents may be prepared by mixing with physiologically acceptable carriers, excipients, or stabilizers in the form of lyophilized powders, slurries, aqueous solutions or suspensions, for example.

The pharmaceutical composition will be formulated for administration to a patient in a therapeutically effective amount sufficient to provide the desired therapeutic result. Preferably, such amount has minimal negative side effects. In one embodiment, the amount of dual cytokine fusion protein administered will be sufficient to treat or prevent inflammatory diseases or condition. In another embodiment, the amount of dual cytokine fusion protein administered will be sufficient to treat or prevent immune diseases or disorders. In still another embodiment, the amount of diakine or dual cytokine fusion protein administered will be sufficient to treat or prevent cancer. The amount administered may vary from patient to patient and will need to be determined by considering the subject's or patient's disease or condition, the overall health of the patient, method of administration, the severity of side-effects, and the like.

An effective amount for a particular patient may vary depending on factors such as the condition being treated, the overall health of the patient, the method route and dose of administration and the severity of side effects. The appropriate dose administered to a patient is typically determined by a clinician using parameters orfactors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

The method for determining the dosing of the presently described dual cytokine fusion protein will be substantially similar to that described in U.S. Pat. No. 10,858,412. Generally, the presently described dual cytokine fusion protein will have a dosing in the range of 0.5 microgram/kilogram to 100 micrograms/kilogram. The dual cytokine fusion protein may be administered daily, three times a week, twice a week, weekly, bimonthly, or monthly. An effective amount of therapeutic will impact the level of inflammation or disease or condition by relieving the symptom. For example, the impact might include a level of impact that is at least 10%; at least 20%; at least about 30%; at least 40%; at least 50%; or more such that the disease or condition is alleviated or fully treated.

Compositions of the application can be administered orally or injected into the body. Formulations for oral use can also include compounds to further protect the variant IL-10 molecules from proteases in the gastrointestinal tract. Injections are usually intramuscular, subcutaneous, intradermal or intravenous. Alternatively, intra-articular injection or other routes could be used in appropriate circumstances. Parenterally administered dual cytokine fusion protein are preferably formulated in a unit dosage injectable form (solution, suspension, emulsion) in association with a pharmaceutical carrier and/or pharmaceutically acceptable excipients. In other embodiments, compositions of the application may be introduced into a patient's body by implantable or injectable drug delivery system.

Testing the Diakine or Dual Cytokine Fusion Protein

A plurality of screening assays are known and available to those of skill in the art to test for the desired biological function. In one embodiment, the desired biological function includes, but are not limited to, reduced anti-inflammatory response, reduce T-cell stimulation, enhanced T-cell function, enhanced Kupffer cell functionality and reduced mast cell degranulation.

For example, it is known that IL-10 exposure primes T cells to generate and secrete more IFNγ upon T cell receptor stimulation. Simultaneously, IL-10 exposure prevents the secretion of TNFα, IL-6 and other pro-inflammatory cytokines secreted from monocytes/macrophages in response to LPS. IL-10 also suppresses FoxP3⁺CD4⁺ T_{reg} proliferation. In one embodiment, the dual cytokine fusion protein that maximize monocyte/macrophage suppression but lack T cell effects, including both stimulatory and suppressive responses, will be positively selected. In one embodiment, screening for dual cytokine fusion proteins that possess increased anti-inflammatory effects will be positively selected for the treatment of autoimmune, anti-inflammatory disease or both. In yet another embodiment, dual cytokine fusion proteins that maximize T cell biology, including both stimulatory and suppressive responses, and also possesses enhanced Kupffer cell scavenging, will be selected to develop for the treatment of cancer. Various assays and methods of screening the dual cytokine fusion proteins are previously described in co-pending U.S. Pat. No. 10,858,412, which is incorporated by reference in its entirety. See, U.S. application Ser. No. 16/811,718 Specification at pages 39-42.

Recombinantly Engineered Cells or ACT

In one aspect, the methods disclosed herein will include the combination of the diakine with an ACT therapy, such as but not limited to the recombinantly engineered cell includes a recombinant antigen receptor in the form of a chimeric antigen receptor (CAR), a TCR, or a functional non-TCR, TILs, NK cells, preferably a CAR-T that targets a TAA associated with a solid tumor. In some embodiments, the antigen receptor comprises an extracellular antigen-recognition domain that specifically binds to a TAA antigen and an intracellular signaling domain.

A typical recombinantly expressed CAR or TCR will comprise as extracellular antigen-recognition domain (“EARD”), a transmembrane domain, and an intracellular domain.

The EARD may be specific for proteins, polypeptides, or carbohydrates. Specifically useful for treating cancers are EARDs that target TAAs. For example, the EARD may target TAAs, such as but not limited to EGFR, VEGFR1, VEGFR2, EGP-2, EGP-4, OEPHa2, ErbB2, 3, or 4, Her2, L1-CAM, CD19, CD20, CD22, mesothelin, CEA, and hepatitis B surface antigen, anti-folate receptor, carcinoembryonic antigen (CEA), prostate specific antigen (PSA), PSMA, Her2/neu, estrogen receptor, progesterone receptor, ephrinB2, CD123, CS-1, c-Met, GD-2, and MAGE A3, CD3, CD4, CD5, CD7, CD23, CD24, CD30, CD33, CD34, CD38, CD40, CD44, CD52, CD56, CD70, CD79B, CD117, CD123, CD138, CD147, BCMA, CLL01, LMP-1, SLAMF7, NY-ESO-1, TCAI, CS-1, CCR4, ROR1, tEGFR, MUC1, MUC16, PSCA, NKG2D Ligands, MART-1, gp100, oncofetal antigen, ROR1, TAG72, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-AL mesothelin, CE7, Wilms Tumor 1 (WT-1), or a cyclin. Those of skill in the art will recognize that the recombinantly engineered cell comprising the EARD will be dictated by the tumor or cancer to be treated. Accordingly, those of skill in the art will be capable of selected the appropriate CAR-T or TCR-T with the appropriate EARD to target the cancer.

Transmembrane domains of the CAR or the TCR are artificial hydrophobic regions or those commonly associated with the EARD or generally well-known transmembrane domains. These include, but are not limited to domains from CD28, CD3 epsilon, CD45, CD4, CD5, CDS, CD9, CD 16, CD22, CD33, CD37, CD64, CD80, CD86, CD 134, CD137, CD 154, or alpha, beta or zeta chain of the TCR.

Those of skill in the art also generally recognize that the intracellular domains of the CAR-T or TCR-T cells will comprise one or more of the following intracellular signaling domains: an ITAM (e.g., CD3-zeta “CD3(”), a costimulatory domain I (“CM1”) (e.g., CD28, CD134, CD137/4-1BB, or ICOS), and/or a costimulatory domain II (“CMII”) (e.g., CD134 or CD137/4-1 BB).

The recombinantly engineered cell may be T-cells, including CD4+ or CD8+ T cells. In other aspects, the T-cells are either autologous or allogenic cells, preferably autologous. In some embodiments, the ratio of CD4+ to CD8+ cells is between about 1:5 and about 5:1. In some embodiments, the ratio of CD4+ to CD8+ cells is between about 1:2 and about 2:1. In some embodiments the dose of cells comprises between about 0.2×106 cells/kg body weight of the subject and about 6×106 cells/kg, about 0.5×106 cells/kg body weight of the subject and about 3×106 cells/kg, between about 0.75×106 cells/kg and about 2.5×106 cells/kg or between about 1×106 cells/kg and about 2×106 cells/kg, each inclusive.

Bispecific Monoclonal Antibodies

In one aspect, the methods disclosed herein will include the combination of a Dikaine with a bispecific monoclonal antibody (BSMab). BSMab are generally known in the art and include, without limitation bispecific T cell Engagers (BiTEs), tandem single chain variable fragments (taFvs), diabodies (Dbs), single chain diabodies (scDbs), triple bodies or trivalent antibodies or fragments thereof, dual-affinity retargeting antibodies (DARTs), or Trident technology. A representative example of a BSMab includes BiTEs, which are broadly described as being a bispecific monoclonal antibody capable of engaging two different antigenic determinants or targets. Conventionally, BiTEs are composed of two scFv molecules wherein the first scFv is capable of recognizing polyclonal immune cells (e.g., CD8+ or CD4+ T-cells or NK cells) and the second scFv is capable of recognizing a tumor antigenic target or TAA. This is generally accomplished by having the first scFv recognizing a CD3 on the surface of a T cell (or NK cells) and the second scFv recognizing a TAA, such as CD20.

In one aspect, the BiTE will include a scFv having specificity for CD3, while the second scFv may have specificity for a variety of TAAs that are found on the surface of both hematological and solid tumors. In one embodiment, the first scFv specific for for CD3 may be combined with a second scFv specific for EGFR, VEGFR1, VEGFR2, EGP-2, EGP-4, OEPHa2, ErbB2, 3, or 4, Her2, L1-CAM, CD19, CD20, CD22, mesothelin, CEA, and hepatitis B surface antigen, anti-folate receptor, carcinoembryonic antigen (CEA), prostate specific antigen (PSA), PSMA, Her2/neu, HER3, estrogen receptor, progesterone receptor, ephrinB2, CD123, CS-1, c-Met, GD-2, and MAGE A3, CD3, CD4, CD5, CD7, CD23, CD24, CD30, CD33, CD34, CD38, CD40, CD44, CD52, CD56, CD70, CD79B, CD117, CD123, CD138, CD147, BCMA, CLL01, LMP-1, SLAMF7, NY-ESO-1, TCAI, CS-1, CCR4, ROR1, tEGFR, MUC1, MUC16, PSCA, NKG2D Ligands, B7-H3, PD-1, PD-L1, EpCAM, PSCA, GPC3, AXL, claudin 18.2, CTLA-4, CEA, PSA, MART-1, gp100, oncofetal antigen, ROR1, TAG72, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-AL mesothelin, CE7, Wilms Tumor 1 (WT-1), or a cyclin. In one aspect, the BSMab (more specifically the BiTE) will have one specificity to a TAA, while the diakine will have specificity for a different TAA. For example, the BiTE may be a BSMab having specificity for CD3 and CD20, while the Dikaine will be in DK2¹⁰ form having a VH and VL scFv specific for CD19.

Methods of Treating and/or Preventing Using the Diakine in Combination with ACT or BiTE

Without being bound to any particular theory, the inventor believes that targeting both IL-10 and IL-2 to the tumor vasculature, such as by directing a diakine to VEGFR2, will enable activation, infiltration and persistence while limiting toxicity both through tumor specific activation of the CAR-T cells and direct suppression of cytokine release syndrome and IL-2 toxicity by DV07 (the high affinity form of IL-10). The inventor also believes that targeting IL-10 alone to the tumor vasculature using a single cytokine version of the fusion protein in Dvegfr2DV07, as described in U.S. Pat. No. 10,858,412, (see also, FIG. 1 as a representative examples of the structure) will also be effective tumor specific activation of the CAR-T cells. In another embodiment, based on similar theories, diakines comprising the combination of IL-10 and IL-7, or IL-12 and IL-10, or IL-10 and IL-15, or, IL-10 and IFN-alpha, or IL-10 and IL-21, or IL-10 and IL-27. In another theory, the inventor believes that diakines in DK2¹⁰ form are capable of priming the immune system to enhance or potentiate the function of conventional ACT or BiTE therapies.

Thus, in one aspect, the present disclosure relates to methods of treating and/or preventing malignant diseases or conditions or cancer comprising administering to a subject in need thereof a therapeutically effective amount of a diakine or a dual cytokine fusion protein comprising IL-10, IL-12, IL-27 and a second cytokine in combination with ACT, such as CAR-T cells or TCR-T cells, or a BSMab, such as a BiTE. Such a dual cytokine fusion protein, as described above, may preferably be in DK2¹⁰ DK7¹⁰, DK12¹⁰, DK15¹⁰, DKIFNa¹⁰, DK21¹⁰, or DK27¹⁰ form, with monomers of DV07 and engrafted with CDRs from any antibody targeting a tumor associated antigen (“TAA”). In a preferred embodiment, the dual cytokine fusion protein is in DK2¹⁰ form comprising DV07. A person of skill in the art would be capable of understanding that the dosage of the diakine or dual cytokine fusion protein described herein may be adjusted as needed or depending on the desired outcome. In one aspect, the diakine is administered to a patient in need thereof at a dose of approximately 0.001 to 0.25 mg/kg, preferably in the dose range of 0.01 to 0.2 mg/kg. In another aspect, the dose administered to the patient in need thereof is sufficient to achieve a serum or plasma concentration of 0.0005 to 250 ng/mL, preferably in the range of 0.001 to 200 ng/mL.

Those of skill in the art will recognize that adoptive cell therapy (such as adoptive T-cell therapy) is well known and practiced according to procedures previously described. See, e.g., U.S. Pat. No. 4,690,915. These methods may include autologous transfer (i.e., derived from the patient) or allogenic transfer (i.e. derived from another subject other than the patient to be treated).

The CAR-T or TCR-T cells are administered by methods known and conventionally practiced by those familiar with adaptive cell therapy. In one embodiment, the administration method includes, but is not limited to bolus infusion, intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, or intralesional or intrtumoral administration. Parenteral infusions include intramuscular, intravenous, intraarterial, intraperitoneal, or subcutaneous administration. In certain some embodiments, the recombinantly engineered CAR-T or TCR-T is administered as a single bolus administration, multiple bolus or continuous infusion.

In one embodiment, the dual cytokine fusion protein and the CAR-T are administered in separate subsequent time periods, wherein, for example, the diakine (such as DK2¹⁰vegfr2) is administered prior to the administration of a recombinantly engineered CAR-T cell. In other embodiments, is administered the dual cytokine fusion protein and the CAR-T are administered simultaneously. In other embodiments, the diakine is administered 1-3 days before the CAR-T therapy and then simultaneously administered along with the CAR-T, and/or 1-7 days following CAR-T administration. The diakine may be administered once a day or week, or 2-3 times a week in combination or conjunction with the CAR-T. In another aspect, the diakine is utilized in the expansion and/or thawing procedure of the CAR-T cells prior to administration. Upon reconstituting CAR-T cells from cryopreserved stock, the CAR-T are typically rested in the presence of CAR-T beneficial cytokines (e.g., low dose IL-2). In one aspect, the CAR-T cells may be primed or expanded from cryopreserved stocks in the presence of a diakine. In one aspect, the CAR-T is expanded or primed in the presence of 0.001 to 300 ng/mL of a diakine, more preferably 0.01 to 200 ng/mL of a diakine.

Likewise, the diakine and the BiTE are administered in separate subsequent time periods, wherein the diakine (e.g., DK2¹⁰CD20) is administered 1-3 days before administering the BiTE (e.g., CD3×CD19 BiTE). In other embodiments, the diakine is administered 1-3 days before the BiTE and then simultaneously administered along with the BiTE, and/or 1-7 days following BiTE administration. The diakine may be administered once a day or week, or 2-3 times a week in combination or conjunction with the BiTE.

In other embodiments, the present disclosure also contemplates methods of co-administration or treatment with a third therapeutic agent, e.g., a cytokine, steroid, chemotherapeutic agent, antibiotic, anti-inflammatory agents, or radiation, are well known in the art. These might include combination treatments with other therapeutic agents, such as but not limited to one or more the following: chemotherapeutics, interferon-β, for example, IFNβ-1α and IFN-β-1 β; a protein that simulates myelin basic protein; corticosteroids; IL-1 inhibitors; TNF inhibitors; anti-TNFα antibodies, anti-IL-6 antibodies, IL-1 br-Ig fusion, anti-IL-23 antibodies, antibodies to CD40 ligand and CD80; antagonists of IL-12 and IL-23, e.g., antagonists of a p40 subunit of IL-12 and IL-23 (e.g., inhibitory antibodies against the p40 subunit); IL-22 antagonists; small molecule inhibitors, e.g., methotrexate, leflunomide, sirolimus (rapamycin) and analogs thereof, e.g., CCI-779; Cox-2 and cPLA2 inhibitors; NSAIDs; p38 inhibitors; TPL-2; Mk-2; NFkβ inhibitors; RAGE or soluble RAGE; P-selectin or PSGL-1 inhibitors (e.g., small molecule inhibitors, antibodies thereto, e.g., antibodies to P-selectin); estrogen receptor beta (ERB) agonists or ERB-NFkβ antagonists.

Additionally, the combination treatment useful for administration with the dual cytokine fusion protein may include TNF inhibitors include, e.g., chimeric, humanized, effectively human, human or in vitro generated antibodies, or antigen-binding fragments thereof, that bind to TNF; soluble fragments of a TNF receptor, e.g., p55 or p75 human TNF receptor or derivatives thereof, e.g., 75 kd TNFR-IgG (75 kD TNF receptor-IgG fusion protein, ENBREL™), p55 kD TNF receptor-IgG fusion protein; and TNF enzyme antagonists, e.g., TNFα converting enzyme (TACE) inhibitors. Other combination treatment with anti-inflammatory agents/drugs that includes, but not limited to standard non-steroidal anti-inflammatory drugs (NSAIDs) and cyclo-oxygenase-2 inhibitors. NSAID may include aspirin, celecoxib, diclofenac, diflunisal, etodolac, ibuprofen, indomethacin, ketoprofen, ketorolac, nabumetone, naproxen, oxaprozin, piroxicam, salsalate, sulindac, and/or tolmetin. The cyclo-oxygenase-2 inhibitor employed in compositions according to the application could, for example, be celecoxib or rofecoxib.

Additional therapeutic agents that can be co-administered and/or co-formulated with the dual cytokine fusion protein include one or more of: interferon-β, for example, IFN β-1α and IFN β-1β; COPAXONE®; corticosteroids; IL-1 inhibitors; TNF antagonists (e.g., a soluble fragment of a TNF receptor, e.g., p55 or p75 human TNF receptor or derivatives thereof, e.g., 75 kdTNFR-IgG; antibodies to CD40 ligand and CD80; and antagonists of IL-12 and/or IL-23, e.g., antagonists of a p40 subunit of IL-12 and IL-23 (e.g., inhibitory antibodies that bind to the p40 subunit of IL-12 and IL-23); methotrexate, leflunomide, and a sirolimus (rapamycin) or an analog thereof, e.g., CCI-779. Other therapeutic agents may include Imfimzi or Atezolizumb.

Representative chemotherapeutic agents that may be co-administered with the dual cytokine fusion protein described herein may include for following non-exhaustive list: include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g. paclitaxel (TAXOL® Bristol-Myers Squibb Oncology, Princeton, N.J.) and doxetaxel (Taxotere™, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; Xeloda® Roche, Switzerland; ibandronate; CPT11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and antiandrogens such as flutamide, nilutamide, bicalutamide, leuprolide, and goserelin; and pharmaceutically acceptable salts, acids or derivatives of any of the above.

### Example 1: Combination of Diakine and CAR-T

Various Effector to Target (E:T) ratios of CAR-T to tumor cells are examined to determine whether combining diakines with CAR-T will enhance the function of CAR-T cytotoxic effects. Briefly, CAR-T cells (Promab) are primed 1-3 days in the presence of diakine (DK2¹⁰CD19) at 0, 10, or 100 ng/mL. Raji cells, which were stably transfected with a constititively expressing GFP, are used as the model tumor cell line to examine CAR-T function. Following priming, the CAR-T cells are mixed at a 3-to-1 or 1-to-3 E:T ratio and allowed to interact with the Raji-GFP cells and monitored on an IncuCyte S3 system (Sartorius). CAR-T cell cytolytic effectiveness is measured by the disappearance of GFP over a period of approximately 48 hours.

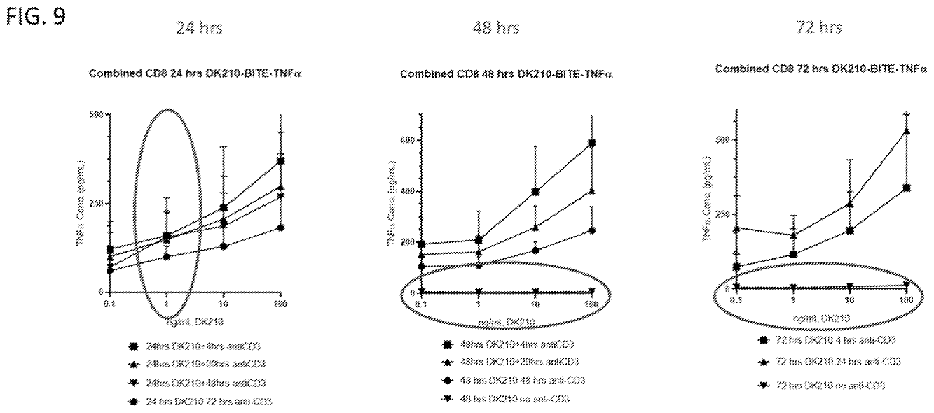

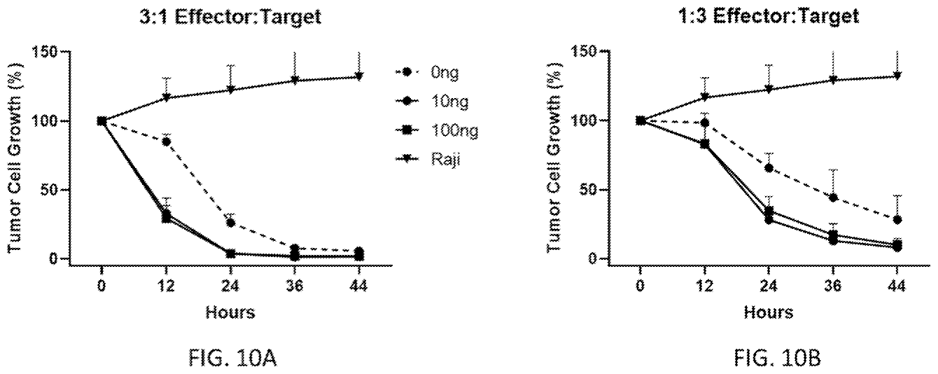

The percentage of tumor cell growth (i.e., disappearance of GFP is a measure that the target cells are being cytolyzed) is measured over time. After 24 hours, the combination of diakine (10 ng/mL or 100 ng/mL) at a 3:1 effector to target ratio of CD20 CAR-T cells to Raji cells exhibited a reduction of tumor cell growth by approximately 6.5-fold when compared to CAR-T cells alone. See, FIG. 9A. Similarly, at 24 hours the combination of diakine (10 ng/mL or 100 ng/mL) at a 1:3 effector to target ratio of CD20 CAR-T cells to Raji cells exhibited a reduction of tumor cell growth by approximately 2-fold when compared to CAR-T cells alone. See, FIG. 10B. These data demonstrate that when diakines are used to prime the CAR-T cells prior to administration, that the CAR-T have an enhanced and more potent cytotoxic function.

### Example 2: Treatment of Solid Tumor with Diakine in Combination with a CAR-T

In this method of treating cancer, the subject is administered an effective amount of a diakine, (e.g., DK2¹⁰vegfr2), which is capable of targeting both IL-10 and IL-2 to the tumor microenvironment, which overexpresses a TAA, such as VEGFR2. Subsequently, using standard protocols of adoptive cell therapy, a CAR-T cell expressing a EARD targeting an over-expressed antigens on tumor cells, such as her2/neu or PSA, is administered. Prior to administering to the patient, the CAR-T cells are expanded, optionally in the presence of diakine. In preparing the engineered cells, the CAR is chosen such that its EARD specifically binds to the antigenic epitope specific for the tumor to be treated.

### Example 3: Combination of Diakine and BiTE

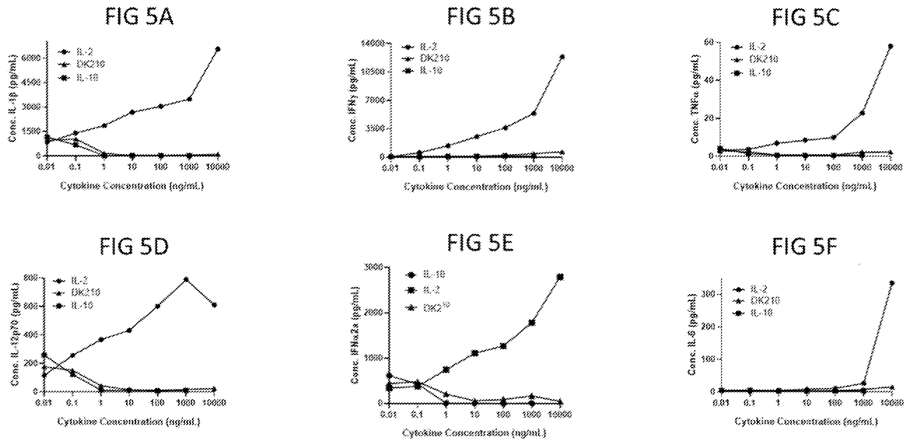

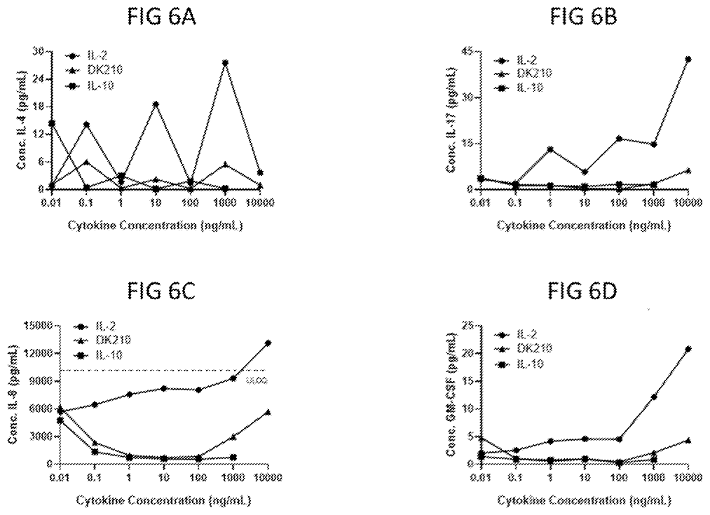

The rationale for determining whether diakines would be beneficial to current BiTEs therapies, was initiated by understanding how diakines behave when exposed to PBMC. Using a model system to determine T cell response (see U.S. Pat. No. 10,858,412 at col. 29-30), PBMC are exposed to various concentrations of diakine (0 ng/mL to 10 ug/mL of DK2¹⁰egfr) followed by an examination of the cytokine induction profile. Naïve PBMC cytokine response profiles are provided in FIG. 5A-5F for IL-1β, IFN-γ, TNF-α, IL-12p70, IFNα2a, and IL6, respectively, and FIG. 6A-6D for IL-4, IL-17, IL-8, and GM-CSF, respectively. These data demonstrate that the diakine configured as DK2¹⁰, which combines IL-10 (DV07) and IL-2 together, dramatically suppressed the induction of cytokines associated with IL-2 alone, making way for a molecule that is capable of enhancing the cytotoxic function without an equally elevated burst of inflammatory cytokines.

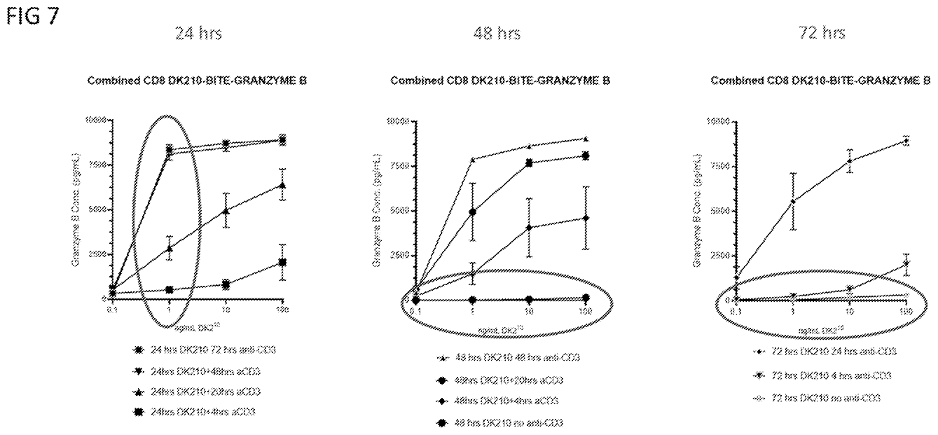

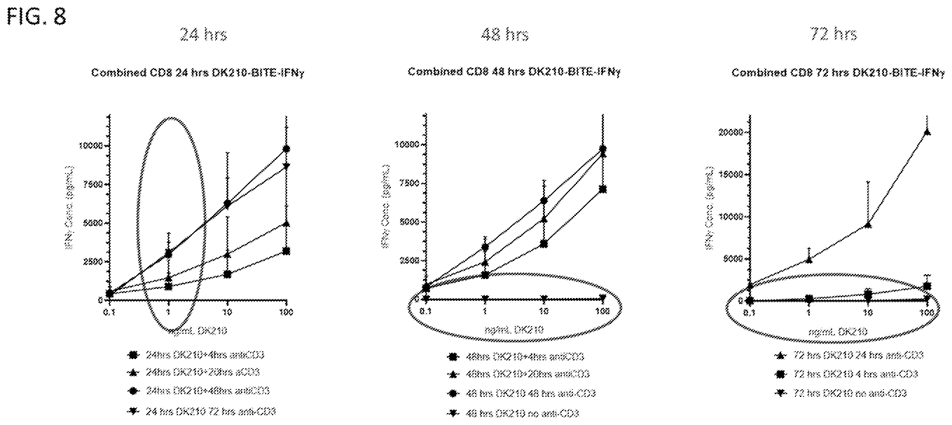

Next, CD8+ T cell response to diakine (DK2¹⁰) priming was assessed. Briefly, CD8+ T cells are magnetically sorted and activated with anti-CD3/anti-CD28 for 3 days. Following activation, the CD8+ T cells are allowed to rest and prime in the presence of a diakine for 24, 48, and 72 hours. Following the rest and priming period (i.e., 24, 48, or 72 hours), levels of Granzyme B, IFN-γ, and TNF-α are evaluated as a result of an anti-CD3 stimulation period for 4, 20, 48, and 72 hours. Priming the CD8+ T cells with as little as 1 ng/mL of a diakine in the DK2¹⁰ form showed elevated levels of Granzyme B (FIG. 7), with concomitantly elevated levels of IFN-γ (FIG. 8), whereas the TNF-α levels (FIG. 9) are suppressed. Taken together, these data demonstrate that when cytotoxic T cells are primed with a diakine in the DK2¹⁰ form, the induction of inflammatory cytokines is relatively suppressed and upon stimulated, the CD8+ T cells become potently active against a target.

Utilizing the rational set forth above, commercially available BiTEs, anti-CD3×anti-CD19 and anti-CD3×anti-CD20, are tested in combination with the diakines DK2¹⁰egfr and DK2¹⁰CD19, respectively. Briefly, CD8+ T cells freshly isolated from healthy donors were assessed for their cytolysis capabilities on Raji cells stably transfected with a constitutively expressing GFP. Raji cells are also previously determined to cell surface express EGFR, CD19, and CD20 (as demonstrated by FACS, data not shown). CD8+ T cells are primed for a period of 1-2 days in the presence of 0 to 100 ng/mL of diakine (DK2¹⁰egfr and DK2¹⁰CD19). BiTE effectiveness to cytolyze Raji cells is measured and monitored on an IncuCyte S3 system (Sartorius) through the disappearance of GFP over a period of approximately 48 hours. Simultaneously, the analyte levels of IFN-γ, TNF-α, granzyme B, and performin, which are the cytokines, proteases, and proteins important for cytotoxic function, are assessed.

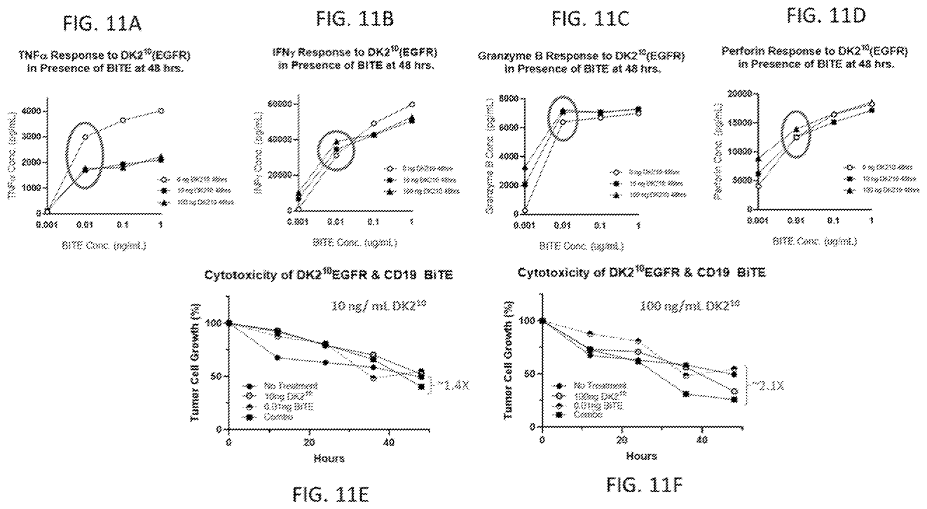

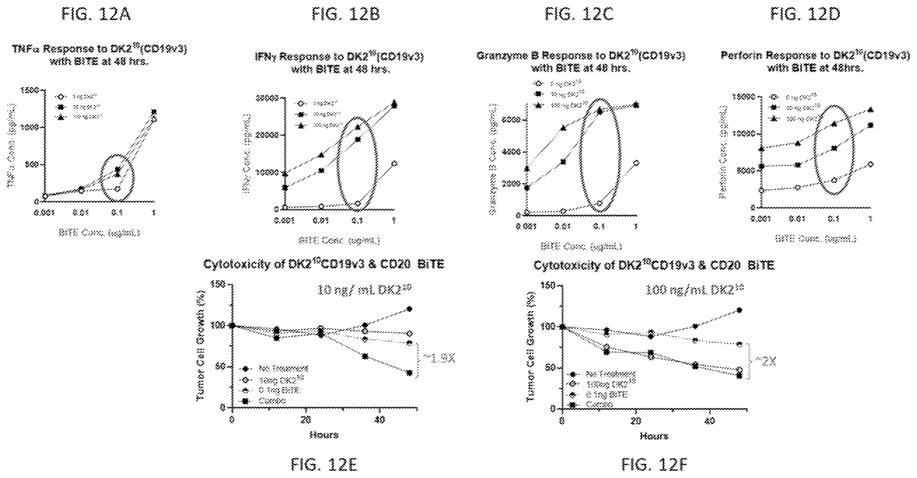

FIG. 11E-11F provide the assessment of combining a diakine in DK2¹⁰egfr form with the lowest functional CD3×CD19 BiTE concentration (0.01 ng/mL) at a 48-hour time point. FIG. 12E-12F provide the assessment of combining a diakine in DK2¹⁰CD19 form with the lowest functional CD3×CD20 BiTE concentration (0.1 ng/mL) at a 48-hour time point. These data suggest that combining diakines DK2¹⁰egfr or DK2¹⁰CD19 with BiTEs, CD3×CD19 or CD3×CD20, respectively, dramatically enhances safety profile (i.e., reducing inflammatory cytokine induction, or enhances cytolytic function (see FIGS. 11A-D and 12A-12D).

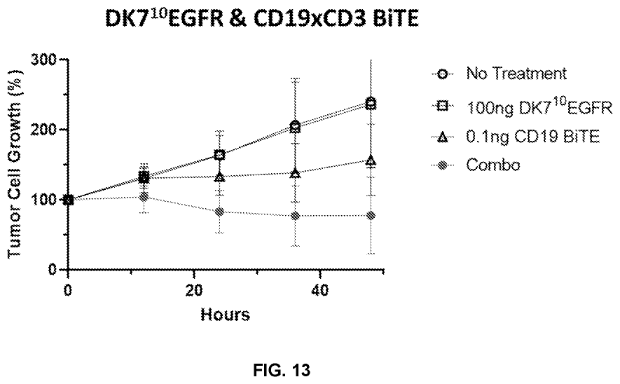

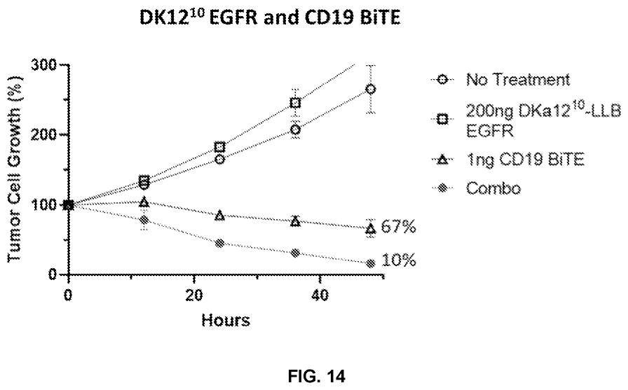

Additional diakine comprising IL-7 and IL-10 (DK7¹⁰) or IL12 and IL-10 (DK12¹⁰) in combination with BiTEs were also assessed using the same assay described above. See FIGS. 13 and 14. These data suggest that combining the diakines of different varieties in combination with BiTEs is effective in enhancing cytolytic function.

### Example 4: Treatment of Tumor with Diakine in Combination with a BiTE

In this method of treating cancer, the subject is administered an effective amount of a diakine in DK2¹⁰ form, which is capable of targeting both IL-10 and IL-2 to a hematological tumor or a tumor microenvironment, which overexpresses the TAA 1-3 days prior to the administration of a BiTE. Subsequently, using standard protocols associated with BiTE administration, a Dikaine is then simultaneously administered to the patient in need thereof along with the BiTE.

This written description uses examples to disclose aspects of the present disclosure, including the preferred embodiments, and also to enable any person skilled in the art to practice the aspects thereof, including making and using any devices or systems and performing any incorporated methods. The patentable scope of these aspects is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. Aspects from the various embodiments described, as well as other known equivalents for each such aspect, can be mixed and matched by one of ordinary skill in the art to construct additional embodiments and techniques in accordance with principles of this application.

### REFERENCES

Assier, E. (2004). NK Cells and Polymorphonuclear Neutrophils Are Both Critical for IL-2-Induced Pulmonary Vascular Leak Sydrome. Journal of Immunology.
Balce, D. R. (2011). Alternative activation of macrophages by IL-4 enhances the proteolytic capacity of their phagosomes through synergistic mechanisms. Blood.
Baluna, R. (1997). Vascular leak syndrome a side effect of immunotherapy. Immunopharmacology.
Bentebibe, S.-E. (2019). A First-in-Human Study and Biomarker Analysis of NKTR-214, a Novel IL2Raf Biased Cytokine, in Patients with Advanced or Metastatic Solid Tumors. Cancer Discovery.
Berman, R. (1996). Systemic administration of cellular IL-10 induces an effective specific and long lived immune response against established tumors in mice. Journal of Immunology.
Bianca Santomasso. (2019). The Other Side of CAR T-Cell Therapy: Cytokine Release Syndrome, Neurologic Toxicity, and Financial Burden. ASCO Publications.
Bonifant, C. L. (2016). Toxicity and management in CAR T-cell therapy. Molecular Therapy.
Buchbinder, E. I. (2019). Therapy with high-dose Interleukin-2 (HD IL-2) in metastatic melanoma and renal cell carcinoma following PD1 or PDL1 inhibition. Journal of Immunotherapy for Cancer.
Castellarin, M. (2018). Driving cars to the clinic for solid tumors. Gene Therapy.
Chan, I. H. (2015). The Potentiation of IFNg and Induction of Cytotoxic Proteins by Pegylated IL-10 in Human CD8 T cells. Journal of Interferon and Cytokine Research.
Chen, X. (2018). A novel human IL-2 mutein with minimal systemic toxicity exerts greater antitumor efficacy than wild-type IL-2. Cell Death and Disease.
Chinen, T. (2016). An essential role for IL-2 receptor in regulatory T cell function. Nature Immunology.
Christopher DeRenzo. (2019). Genetic Modification Strategies to Enhance CART Cell Persistence for Patients With Solid Tumors.
Davis, I. D. (2009). A Phase I and Pharmacokinetic Study of Subcutaneously-Administered Recombinant Human Interleukin-4 (rhuIL-4) in Patients with Advanced Cancer. Growth Factors.
Emmerich, J. (2012). IL-10 Directly Activates and Expands Tumor-Resident CD80 T Cells without DeNovo Infiltration from Secondary Lymphoid Organs. Cancer Research, 3570-3581.
Fedorak, R. (2000). Recombinant Human Interlekin 10 in the Treatment of Patients with Mild to Moderately Active Crohn's Disease. Gastroenterology, 1473-1482.
Gooch, J. L. (1998). Interleukin 4 Inhibits Growth and Induces Apoptosis in Human Breast Cancer Cells. Cancer Research.
Greve, J. M. (2000). U.S. Pat. No. 6,028,176.
Groux, H. (1998). Inhibitory and Stimulatory Effects of IL-10 on Human CD8+ T cells. The Journal of Immunology.
Guan, H. (2007). Blockade of Hyaluronan Inhibits IL-2 Induced Vascular Leak Syndrome and Maintains Effectiveness of IL-2 Treatment in Metastatic Melanoma. Journal of Immunology.
Hart, P. H. (1989). Potential antiinflammatory effects of interleukin 4: Suppression of human monocyte tumor necrosis factor ca, interleukin 1, and prostaglandin E2. PNAS.
Hart, P. H. (1991). IL-4 suppresses IL-1, TNF-a and PGE2 production by human peritoneal macrophages. Immunology.
Jafarzadeh, L. (2020). Prolonged Persistence of Chimieric Antigen Receptor CAR T Cell in Adoptive Cancer Immunotherapy Challenges and Ways Forward. Frontiers in Immunology.
Jiang, T. (2016). Role of IL-2 in cancer immunotherapy. Oncoimmunology.
Kirchner, G. I. (1998). Pharmacokinetics of human Interleukin-2 in advanced renal cell carcinoma patients following subcutaneous application. British Journal Clinical Pharmacology.
Lee, H. L. (2016). Tumor growth suppressive effect of IL-4 through p21-mediated activation of STAT6 in IL-4Ra overexpressed melanoma models. Oncotarget.
Li, R. (2013). Expression of recombinant human IL-4 in Pichia pastoris and relationship between its glycosylation and biological function. Protein Expression and Purification.
Ma, S. (2019). Current Progress in CAR-T Cell Therapy for Solid Tumors. International Journal of Biological Sciences.
Malefyt, R. d. (1991). Interleukin 10 inhbits cytokine synthesis by human monocytes: An autoreglatory role of IL-10 produced by monocytes. JEM.
Malefyt, R. d. (1991). Interleukin 10 Inhibits Cytokine Synthesis by Human Monocytes An Autoregulatory Role of IL-10 Produced by Monocytes. Journal of Experimental Medicine, 1209-1220.
McCauley, S. (2018). Pegilodecakin, a Pegylated Human IL-10 (AM0010), Enhances the Cytotoxicity of CAR-T Cells in Vitro and In Vivo. Blood (p. 703). American Society of Hematology.
McGuirk, P. (2000). A Regulatory Role for Interleukin 4 in Differential Inflammatory Responses in the Lung following Infection of Mice Primed with Th1- or Th2-Inducing Pertussis Vaccines. Infection and Immunity.
Moore, K. W. (2001). Interleukin 10 and the Interleukin 10 Receptor. Annual Reviews Immunology.
Mumm, J. (2011). IL-10 induces IFNg-Mediated Tumor Immunity. Cancer Cell.
Mumm, J. B. (2011). IL-10 Elicits IFNg-Dependent Tumor Immune Surveillance. Cancer Cell.
Naing, A. (2016). Safety, Antitumor Activity, and Immune Activation of Pegylated Recombinant Human Interleukin-10 (AM0010) in Patients With Advanced Solid Tumors. Journal of Clinical Oncology.
Naing, A. (2016). Safety, Antitumor Activity, and Immune Activation of Pegylated Recombinant Human Interleukin-10 (AM0010) in Patients With Advanced Solid Tumors. Journal of Clinical Oncology.
Naing, A. (2018). PEGylated IL-10 (Pegilodecakin) Induces Systemic Immune Activation, CD8+ T cell Invigoration and Polyclonal T cell Expansion in Cancer Patients. Cancer Cell.
Rodriguez-Garcia, A. (2020). CART Cells Hit the Tumor Microenvironment Strategies to Overcome Tumor Escape. Frontiers in Immunology.
Ross, S. H. (2018). Signaling and Function of Interleukin-2 in T Lymphocytes. Annual Review Immunology.
Ryan, J. J. (1997). Interleukin-4 and its receptor: Essential mediators of the allergic response. The Journal of Allergy and Clinical Immunology.
Schreiber, S. (2000). Safety and Efficacy of Recombinant Human Interleukin 10 in Chronic Active Crohn's Disease. Gastroenterology, 1461-1472.
Scott, M. J. (2006). Interleukin-10 suppresses natural killer cell but not natural killer T cell activation during bacterial infection. Cytokine.
Sivakumar, P. V. (2013). Comparison of Vascular Leak Syndrome in Mice Treated with IL21 or IL2. Comparative Medicine.
Smith, N. R. (2010). Vascular Endothelial Growth Factor Receptors VEGFR2 and VEGFR-3 Are Localized Primarily to the Vasculature in Human Primary Solid Cancers. Human Cancer Biology.
Spigel, D. R. (2020). Randomized phase II study of pembrolizumab (P) alone versus pegilodecakin (PEG) in combination with P as first-line (1 L) therapy in patients (pts) with stage IV non-small cell lung cancer (NSCLC) with high PD-L1 expression (CYPRESS 1). ASCO, (p. 9563).
Steinke, J. W. (2001). Th2 cytokines and asthma Interleukin-4: its role in the pathogenesis of asthma, and targeting it for asthma treatment with interleukin-4 receptor antagonists. Respiratory Research.
Tang, A. (2018). The challenges and molecular approaches surrounding interleukin-2-based therapeutics in cancer. Cytokine.
Varin, A. (2010). Alternative activation of macrophages by IL-4 impairs phagocytosis of pathogens but potentiates microbial-induced signalling and cytokine secretion. Blood.
Wagner, J. (2020). CAR T Cell Therapy for Solid Tumors: Bright Future or Dark Reality?Molecular Therapy.
Woodward, E. A. (2012). The anti-inflammatory actions of IL-4 in human monocytes are not mediated by IL-10, RP105 or the kinase activity of RIPK2. Cytokine.
Zou, F. (2019). Engineered triple inhibitory receptor resistance improves anti-tumor CAR-T cell performance via CD56. Nature Communications.

1. A method of treating a patient with a cancer and/or a tumor comprising administering to the patient in need thereof, a therapeutically effective amount of a targeted diakine and a genetically modified immune cell expressing a chimeric antigen receptor (CAR) T-cell
wherein the targeted diakine comprises IL10, IL2, and a first single chain variable fragment (scFv) with specificity to a first tumor associated antigen (TAA), the TAA being selected from CD3, CD4, CD5, CD7, CD19, CD20, CD22, CD25, CD30, CD33, CD34, CD38, CD40, CD52, CD56, CD70, CD79B, CD117, CD123, CD138, CD147, B cell maturation antigen (BCMA), C-type lectin-like molecule-1 (CLL01), latent membrane protein 1 (LMP-1), signaling lymphocytic activation molecule F7 (SLAMF7), NY-ESO-1, transmembrane activator and CAML interactor (TACI), CS-1, CXCR4, NKG2D, B7-H3, EGFR, PD-1, PDL-1, HER2, HER3, EpCAM, mesothelin, PSCA, MUC1, Lewis-Y, GPC3, AXL, Claudin18.2, GD2, CTLA-4, CEA, PDGFR, VEGFR2, mesothelin (MESO), PSCA, PSA
and wherein the CAR T-cell comprises a second scFv having specificity to a second and different TAA.

2. The method according to claim 1, wherein the cancer is a hematologic cancer.

3. The method according to claim 2, wherein the hematologic cancer is a lymphoma or leukemia.

4. The method according to claim 2, wherein the hematologic cancer is B cell acute lymphocytic leukemia (B-ALL), multiple myeloma (MM), B cell lymphoma, chronic lymphocytic leukemia (CLL), T-cell Acute Lymphoblastic Leukemia, or Non-Hodgkin Lymphoma.

5. The method according to claim 1, wherein the cancer is a solid cancer or tumor.

6. The method according to claim 5, wherein the solid cancer is neuroblastoma, small cell lung cancer, melanoma, ovarian cancer, renal cell carcinoma, colon cancer, lung cancer, melanoma, breast cancer, prostate cancer, colon cancer, renal cell carcinoma, ovarian cancer, neuroblastoma, rhabdomyosarcoma, leukemia and lymphoma.

7. The method according to claim 1, wherein the IL10 is a human IL10, EBV IL10, or CMV IL10, or muteins or variants thereof and wherein L2 is a wild-type IL2 or a variant, or a mutein thereof.

8. The method according to claim 1, wherein the diakine comprises an IL10 of SEQ ID No: 1, 3, 5 or 7.

9. The method according to claim 1, wherein the diakine and the CAR T-cell are administered to the patient at separate times.

10. The method according to claim 1, wherein the CAR T-cell is thawed and/or rested in the presence of an amount of diakine prior to administration to the patient.

11. The method according to claim 11, wherein the diakine is administered to the patient 1-3 days before administering the CAR T-cell.

12. The method according to claim 11, wherein the diakine and the CAR T-cell are simultaneously administered to the patient.

13. The method according to claim 11, wherein the diakine is administered to the patient 1-3 days before administering the CAR T-cell and then readministering the diakine to the patient 1-3 days after administering the CAR T-cell.

14. The method according to claim 1, wherein the diakine is administered at a dose concentration range of 0.01 to 0.2 mg/kg.

15. The method according to claim 1, wherein the diakine is administered at a dose that achieves a serum or plasma concentration of about 0.0001 to 200 ng/mL.

16. The method according to claim 1, wherein the diakine is administered subcutaneously or intravenously.

17. The method according to claim 1, wherein the genetically modified cell is an immune cell.

18. The method according to 1, wherein the genetically modified cell is a CD8+, CD4+, or a combination thereof.

19. A method of priming a CAR-T cell comprising contacting the CAR-T cell in the presence of 0.01 to 200 ng/mL of a diakine.

20. The method according to claim 19, wherein the CAR-T cells comprise both CD8+ and CD4+ T cells.

21. The method according to claim 19, wherein the CAR-T is primed in the presence of the diakine for a period of 1-2 days prior to administration to a patient in need thereof.

22. A method of treating a patient with a cancer and/or a tumor comprising administering to the patient in need thereof, a therapeutically effective amount of a targeted diakine and a bispecific T cell engager (BiTE), wherein the diakine
wherein the targeted diakine comprises IL10, IL2, and a first single chain variable fragment (scFv) with specificity to a first tumor associated antigen (TAA), the TAA being selected from CD4, CD5, CD7, CD19, CD20, CD22, CD25, CD30, CD33, CD34, CD38, CD40, CD52, CD56, CD70, CD79B, CD117, CD123, CD138, CD147, B cell maturation antigen (BCMA), C-type lectin-like molecule-1 (CLL01), latent membrane protein 1 (LMP-1), signaling lymphocytic activation molecule F7 (SLAMF7), NY-ESO-1, transmembrane activator and CAML interactor (TACI), CS-1, CXCR4, NKG2D, B7-H3, EGFR, PD-1, PDL-1, HER2, HER3, EpCAM, mesothelin, PSCA, MUC1, Lewis-Y, GPC3, AXL, Claudin18.2, GD2, CTLA-4, CEA, PDGFR, VEGFR2, mesothelin (MESO), PSCA, or PSA, and
wherein the BiTE comprises at least a first antigen binding specificity for CD3 and a second antigen binding specificity for a TAA that is different from TAA binding specificity of the diakine scFv.

23. The method according to claim 24, wherein the cancer is a hematologic cancer.

24. The method according to claim 25, wherein the hematologic cancer is a lymphoma or leukemia.

25. The method according to claim 25, wherein the hematologic cancer is B cell acute lymphocytic leukemia (B-ALL), multiple myeloma (MM), B cell lymphoma, chronic lymphocytic leukemia (CLL), T-cell Acute Lymphoblastic Leukemia, or Non-Hodgkin Lymphoma.

26. The method according to claim 24, wherein the cancer is a solid cancer or tumor.

27. The method according to claim 28, wherein the solid cancer is neuroblastoma, small cell lung cancer, melanoma, ovarian cancer, renal cell carcinoma, colon cancer, lung cancer, melanoma, breast cancer, prostate cancer, colon cancer, renal cell carcinoma, ovarian cancer, neuroblastoma, rhabdomyosarcoma, leukemia and lymphoma.

28. The method according to claim 24, wherein the IL10 is a human IL10, EBV IL10, or CMV IL10, or variants and/or muteins thereof.

29. The method according to claim 24, wherein the diakine comprises an IL10 of SEQ ID No: 1, 3, 5, or 7.

30. The method according to claim 24, wherein the IL2 is human.

31. The method according to claim 32, wherein the IL2 is a wild-type IL2 or a variant, or a mutein thereof.

32. The method according to claim 24, wherein the diakine and the BiTE are administered to the patient at separate times.

33. The method according to claim 34, wherein the diakine is administered to the patient 1-3 days before administering the BiTE.

34. The method according to claim 34, wherein the diakine and the BiTE are simultaneously administered to the patient.

35. The method according to claim 34, wherein the diakine is administered to the patient 1-3 days before administering the BiTE and then readministering the diakine to the patient 1-3 days after administering the BiTE.

36. The method according to claim 24, wherein the diakine is administered at a dose concentration range of 0.001 to 200 mg/kg.

37. The method according to claim 24, wherein the diakine is administered at a dose that achieves a serum or plasma concentration of about 0.0001 to 200 ng/mL.

38. The method according to claim 24, wherein the diakine is administered subcutaneously or intravenously.

39. A method of treating a tumor in a patient comprising administering dual cytokine fusion protein of formula (I)
NH₂-(IL10)-(X¹)—(Zₙ)—(X²)—(IL10)-COOH (Formula I);
wherein
“IL-10” is a monomer sequence selected from SEQ ID Nos: 1, 3, 9, 10, 11, 12, 14, or 16;
“X¹” is a VL or VH region obtained from a first monoclonal antibody;
“X²” is a VH or VL region obtained from the first monoclonal antibody;
wherein when X¹ is a VL, X² is a VH or when X¹ is a VH, X² is a VL
“Z” is a cytokine other than IL-10;
“n” is an integer selected from 0-2; and
a recombinantly engineered CAR-T or TCR-T cell specific for an antigen expressed by the cancer or the tumor.

40. The method according to claim 42, wherein X¹ and X² are obtained from the first monoclonal antibody specific for epidermal growth factor receptor (EGFR); CD14; CD52; various immune check point targets, such as but not limited to PD-L1, PD-1, TIM3, BTLA, LAG3 or CTLA4; CD19, CD20, CD22; CD47; GD-2; VEGFR1, VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; SR-J1; HIV, or Ebola.

41. The method according to claim 42, wherein the VL and VH are obtained from the first monoclonal antibody that is an anti-HIV or anti-Ebola antibody.

42. The method according to claim 44, wherein the VL and VH from the anti-HIV or anti-Ebola monoclonal antibody is a framework region that is engraft able with 6 CDRs from a second antibody.

43. The method according to claim 45, wherein the second antibody is a monoclonal antibody selected from epidermal growth factor receptor (EGFR); CD14; CD52; various immune check point targets, such as but not limited to PD-L1, PD-1, TIM3, BTLA, LAG3 or CTLA4; CD20; CD47; GD-2; VEGFR1; VEGFR2; HER2; PDGFR; EpCAM; ICAM (ICAM-1, -2, -3, -4, -5), VCAM, FAPα; 5T4; Trop2; EDB-FN; TGFβ Trap; MAdCAM, β7 integrin subunit; α4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; or SR-J1.

44. The method according to claim 46, wherein the 6 engrafted CDRs from the second monoclonal antibody comprise 6 CDRs from an anti-EGFR antibody, an anti-HER2 antibody, an anti-VEGFR1 antibody, or an anti-VEGFR2 antibody wherein the 6 CDRs comprise CDR 1-3 from the VL and CDR 1-3 from VH.

45. The method according to claim 42, wherein Z is a cytokine selected from IL-6, IL-4, IL-1, IL-2, IL-3, IL-5, IL-7, IL-8, IL-9, IL-15, IL-21, IL-17, IL-26, IL-27, IL-28, IL-29, GM-CSF, G-CSF, TSLP, interferons-α, -β, -γ, TGF-β, or tumor necrosis factors-α, -β, basic FGF, EGF, PDGF, IL-4, IL-11, or IL-13.

46. The method according to claim 42, wherein Z is a IL-2.

47. The method according to claim 42, wherein Z is an integer of 1.

48. The method according to claim 42, wherein the dual cytokine fusion protein further comprises linkers.

49. The method according to claim 42, wherein the IL-10 is DV07 of SEQ ID No: 10.

50. The method according to claim 42, wherein the VH and VL regions are obtained from a first antibody that is a human anti-ebola antibody; the VH and VL regions are engrafted with 6 CDRs from an anti-VEGFR2 antibody; and the Z is IL-2.

51. The method according to claim 42, wherein the recombinantly engineered CAR-T comprises an EARD that is specific for EGFR, VEGFR1, VEGFR2, EGP-2, EGP-4, OEPHa2, ErbB2, 3, or 4, Her2, L1-CAM, CD19, CD20, CD22, mesothelin, CEA, and hepatitis B surface antigen, anti-folate receptor, carcinoembryonic antigen (CEA), prostate specific antigen (PSA), PSMA, Her2/neu, estrogen receptor, progesterone receptor, ephrinB2, CD123, CS-1, c-Met, GD-2, and MAGE A3, CD23, CD24, CD30, CD33, CD38, CD44, ROR1, tEGFR, MUC1, MUC16, PSCA, NKG2D Ligands, NY-ESO-1, MART-1, gp100, oncofetal antigen, ROR1, TAG72, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-AL mesothelin, CE7, Wilms Tumor 1 (WT-1), or a cyclin.
